(19) United States
(12) Patent Application Publication
(10) Pub. No.: US 2021/0065847 A1
(43) Pub. Date: Mar. 4, 2021

(54) SYSTEMS AND METHODS FOR DETERMINING CONSENSUS BASE CALLS IN NUCLEIC ACID SEQUENCING

(71) Applicant: GRAIL, Inc., Menlo Park, CA (US)

(72) Inventors: Anton Valouev, Palo Alto, CA (US); Shirley Chen, San Jose, CA (US); David Burkhardt, San Francisco, CA (US); Christopher Chang, Mountain View, CA (US)

(21) Appl. No.: 17/006,124

(22) Filed: Aug. 28, 2020

Related U.S. Application Data
(60) Provisional application No. 62/894,206, filed on Aug. 30, 2019.

(51) Int. Cl. G16B 30/10; G16B 40/00

(52) U.S. Cl. CPC G16B 30/10; G16B 40/00

(57) ABSTRACT

Systems and methods for determining consensus base calls in nucleic acid sequencing are provided. A sequencing dataset is obtained corresponding to a plurality of base reads for a first base position within a plurality of base positions of a target nucleic acid molecule. The sequencing dataset includes at least two features, for each base read of the plurality of base reads. The at least two features are selected from among the features: a nucleotide base, a read quality score, a strand identifier, a trinucleotide context of the base read, and a confidence score associated with the trinucleotide context. The sequencing dataset is transformed into a feature tensor representing a distribution of the plurality of features in the sequencing dataset. The feature tensor is assessed with a classifier to determine a consensus base call for the first base position. The consensus base call comprises a predicted nucleotide base.

### CROSS REFERENCE TO RELATED APPLICATION

This application claims priority to U.S. Provisional Patent Application No. 62/894,206 entitled “SYSTEMS AND METHODS FOR DETERMINING CONSENSUS BASE CALLS IN NUCLEIC ACID SEQUENCING,” filed Aug. 30, 2019, which is hereby incorporated by reference.

### TECHNICAL FIELD

This specification describes using features of a plurality of base reads to determine a consensus base call for a base position in a nucleic acid.

### BACKGROUND

The increasing knowledge of the molecular basis for cancer and the rapid development of next generation sequencing techniques are advancing the study of early molecular alterations involved in cancer development in body fluids. Large-scale sequencing technologies, such as next generation sequencing (NGS), have afforded the opportunity to achieve sequencing at costs that are less than one U.S. dollar per million bases, and costs of less than ten U.S. cents per million bases have been realized. Specific genetic and epigenetic alterations associated with such cancer development are found in plasma, serum, and urine cell-free DNA (cfDNA). Such alterations could potentially be used as diagnostic biomarkers for several classes of cancers (see, Salvi et al., 2016, Onco Targets Ther. 9:6549-6559).

Cell-free DNA (cfDNA) can be found in serum, plasma, urine, and other body fluids (Chan et al., 2003, Ann Clin Biochem. 40(Pt 2):122-130) representing a “liquid biopsy,” which is a circulating picture of a specific disease (see, De Mattos-Arruda and Caldas, 2016, Mol Oncol. 10(3):464-474). This represents a potential, non-invasive method of screening for a variety of cancers.

The existence of cfDNA was demonstrated by Mandel and Metais decades ago (Mandel and Metais, 1948, C R Seances Soc Biol Fil. 142(3-4):241-243). cfDNA originates from necrotic or apoptotic cells, and it is generally released by all types of cells. Stroun et al. further showed that specific cancer alterations could be found in the cfDNA of patients (see, Stroun et al., 1989 Oncology 1989 46(5):318-322). A number of subsequent articles confirmed that cfDNA contains specific tumor-related alterations, such as mutations, methylation, and copy number variations (CNVs), thus confirming the existence of circulating tumor DNA (ctDNA) (see Goessl et al., 2000 Cancer Res. 60(21):5941-5945 and Frenel et al., 2015, Clin Cancer Res. 21(20):4586-4596).

cfDNA in plasma or serum is well characterized, while urine cfDNA (ucfDNA) has been traditionally less characterized. However, recent studies demonstrated that ucfDNA could also be a promising source of biomarkers (e.g., Casadio et al., 2013, Urol Oncol. 31(8):1744-1750).

In blood, apoptosis is a frequent event that determines the amount of cfDNA. In cancer patients, however, the amount of cfDNA seems to be also influenced by necrosis (see, Hao et al., 2014, Br J Cancer 111(8):1482-1489 and Zonta et al., 2015, Adv Clin Chem. 70:197-246). Since apoptosis seems to be the main release mechanism circulating cfDNA has a size distribution that reveals an enrichment in short fragments of about 167 bp, (see, Heitzer et al., 2015, Clin Chem. 61(1):112-123 and Lo et al., 2010, Sci Transl Med. 2(61):61ra91) corresponding to nucleosomes generated by apoptotic cells.

The amount of circulating cfDNA in serum and plasma seems to be significantly higher in patients with tumors than in healthy controls, especially in those with advanced-stage tumors than in early-stage tumors (see Sozzi et al., 2003, J Clin Oncol. 21(21):3902-3908, Kim et al., 2014, Ann Surg Treat Res. 86(3):136-142; and Shao et al., 2015, Oncol Lett. 10(6):3478-3482). The variability of the amount of circulating cfDNA is higher in cancer patients than in healthy individuals (see, Heitzer et al., 2013, Int J Cancer. 133(2):346-356), and the amount of circulating cfDNA is influenced by several physiological and pathological conditions, including proinflammatory diseases (see, Raptis and Menard, 1980, J Clin Invest. 66(6):1391-1399, and Shapiro et al., 1983, Cancer 51(11):2116-2120).

Methylation status and other epigenetic modifications are known to be correlated with the presence of some disease conditions such as cancer (see, Jones, 2002, Oncogene 21:5358-5360). And specific patterns of methylation have been determined to be associated with particular cancer conditions (see Paska and Hudler, 2015, Biochemia Medica 25(2):161-176). Warton and Samimi have demonstrated that methylation patterns can be observed even in cell free DNA (Warton and Samimi, 2015, Front Mol Biosci, 2(13) doi: 10.3389/fmolb.2015.00013).

Detection of all these genomic modifications, which is important for providing accurate diagnoses to patients, depends upon accurate sequencing, both in terms of sequence alignment and base calling. See e.g., Schmitt et al., 2012, PNAS 109(36), 14508-14513. Next-generation sequencing techniques have a relatively high sequencing error rate that can lead to misidentifying genomic variants. See e.g., Bobo et al., 2016 BioRxiv doi:10.1101/066043. As discussed by Ruffalo et al., mapping accuracy of many genome assembly methods falls far below theoretical accuracies (see 2012, ECCB 28, i349-i355).

Given the role of genomic variants in determining personalized cancer treatments, improved ways of nucleic acid sequencing that enable accurate sequencing and detection of variants are needed in the art.

### SUMMARY

The present disclosure addresses the shortcomings identified in the background by providing robust techniques for determining consensus base calls in nucleic acid sequencing.

In one aspect, a method of determining a consensus base call for a base position within a plurality of base positions of a target nucleic acid molecule in a biological sample of a subject is provided. The method comprises (a) obtaining a sequencing dataset, corresponding to a plurality of base reads for a first base position within the plurality of base positions of the target nucleic acid molecule. Each base read of the plurality of base reads is captured in a respective sequence read among the plurality of sequence reads of the target nucleic acid molecule. The sequencing dataset includes features for each base read of the plurality of base reads. Each base read includes at least two features from the group consisting of: (1) a nucleotide base of the base read, (2) a read quality score associated with the base read, (3) a strand identifier indicating whether the respective read of the base read corresponds to a forward or a reverse strand of the target nucleic acid molecule, (4) a trinucleotide context based on three consecutive nucleotide bases including the base read, and (5) a confidence score associated with the trinucleotide context. In some embodiments, additional features are included for each base read beyond these enumerated features. The method further comprises (b) transforming the sequencing dataset into a feature tensor that represents a distribution of the plurality of features in the sequencing dataset, and (c) assessing the feature tensor with a classifier to determine the consensus base call for the first base position. Each consensus base call comprises a predicted nucleotide base.

In some embodiments, the method further comprises d) repeating the obtaining (a), transforming (b), and assessing (c) for at least 10 base positions, at least 100 base positions, at least 1000 base positions, at least 10,000 base positions, at least 100,000 base positions, or at least 1 million base positions. In some embodiments, the method is performed within one hour or less, thirty minutes or less, ten minutes or less, five minutes or less, or 1 minute or less. For instance, in some embodiment, 100,000 based positions are assessed in accordance with the method in one hour or less, thirty minutes or less, ten minutes or less, five minutes or less, or 1 minute or less.

In some embodiments, the classifier determines a model quality score for the consensus base call. In some embodiments, the model quality score is of the form:

Q=−10·log₁₀(Pₑᵣᵣₒᵣ).

In some embodiments, Pₑᵣᵣₒᵣ is a probability that the predicted nucleotide base is incorrect and is of the form: Pₑᵣᵣₒᵣ=1−P_{c}. In some embodiments, P_{c} is based on a class probability of the predicted nucleotide base computed by the classifier.

In some embodiments, the method further comprises recalibrating the model quality score based on an empirical error rate. In some embodiments, recalibrating the model quality score comprises identifying a quality bin corresponding to the model quality score and interpolating the model quality score based on an actual error rate at the quality bin to generate a recalibrated model quality score. In some embodiments, the quality bin is one of a plurality of quality bins defined by model quality scores obtained during training of the classifier with training data. In some embodiments, the actual error rate is a misclassification rate of the classifier on the training data.

In some embodiments, transforming the sequencing dataset into the feature tensor comprises converting the features in the first sequencing dataset into a multidimensional array, and flattening the multidimensional array into a one-dimensional vector.

In some embodiments, the feature tensor represents a quantified distribution of the features in the sequencing dataset. In some embodiments, the quantified distribution is determined by sorting each base read in the plurality of base reads by rank, generating a value for each base read in accordance with its respective rank, and representing the value at each base read in the feature tensor.

In some embodiments, the features for each base read comprise a respective nucleotide base and a respective read quality score. In such embodiments, the quantified distribution comprises a discounted distribution that is determined by: (i) ranking the plurality of base reads by order of decreasing respective read quality scores such that each base read in the plurality of base reads has a respective rank number “k,” (ii) generating the value for each base read with a discounted value based on its respective rank number “k,” and (iii) representing the discounted value of each base read at an element in the feature tensor that coincides with a respective nucleotide base and a respective read quality score of the base read. In some embodiments, for each element coinciding with a base read, the element with the discounted value of the base read is assigned. In some embodiments, for each element coinciding with a plurality of base reads, the element with a sum of the discounted values of the plurality of base reads is assigned, and for each element coinciding with no base reads, the element with a zero or null value is assigned.

In some embodiments, assessing the feature tensor with the classifier comprises computing class probabilities based on the feature tensor and determining the consensus base call based on a highest class probability among the class probabilities. In some embodiments, each of the class probabilities corresponds to a set of classes that includes at least four classes selected from the group consisting of: adenine (“A”), guanine (“G”), thymine (“T”), cytosine (“C”), and uracil (“U”).

In some embodiments, the classifier comprises a multinomial logistic regression model and a set of model coefficients learned during training of the classifier.

In some embodiments, the method further comprises training the classifier with a penalized logistic regression model to derive model coefficients for predicting a nucleotide base and a model quality score from the features selected from the group. In some embodiments, the penalized logistic regression model is selected based on principal component analysis of training data and somatic and germline mutations are removed from the training data.

In some embodiments, the features for determining the consensus base call are selected based on principal component analysis of misclassified examples of training data.

In some embodiments, the features for determining the consensus base call are selected based on principal component analysis of misclassified examples of training data. In some embodiments, the features include additional features for improving classification.

In some embodiments, the features also include additional features for each base read at the first base position. In some embodiments, the additional features comprise at least one feature selected from the group consisting of: (1) a bag depth count of a total number of base reads at the first base position, (2) a first bag depth indicating when the total number of base reads is below a minimum threshold, (3) a distance of the base read relative to an end of its respective sequence read, (4) a terminal base (e.g., a position at an edge of the respective sequence read) indicating whether the base read is an edge or a non-edge base, (5) a duplex status indicating whether the base read is associated with a duplex or non-duplex read of the first base position, (6) a certain trinucleotide context indicating when the base read is one of three predetermined adjacent nucleotide bases, (7) an overlap status indicating whether the base read is a stitched or non-stitched read, (8) an ambiguous strand base count at the base read, (9) a mapping quality of the base read, (10) a distance of the base read from a homopolymer, insertion, or deletion, (11) one or more interaction features between or within duplex strands, and (12) a read direction indicating whether the respective sequence read of the base read is a forward or reverse read of the target nucleic acid molecule.

In some embodiments, determining the feature tensor comprises representing values associated with the additional features at one or more elements appended at an end of the feature tensor.

In some embodiments, the plurality of sequence reads defines a pile up sequence reads having the same unique molecule identifier or the same genomic position.

In some embodiments, the method further comprises determining a plurality of consensus base calls for a plurality of base positions of the target nucleic acid molecule to generate the consensus sequence.

In some embodiments, the sequencing dataset is obtained from a methylation sequencing. In some embodiments, the sequencing dataset is obtained from a targeted sequencing.

In some embodiments, the method further comprises, prior to the obtaining the sequencing dataset, obtaining a mapping string for each sequence read in a plurality of sequence reads of the target nucleic acid molecule, thereby obtaining a plurality of mapping strings, where each mapping string in the plurality of mapping strings is determined by an alignment of the respective sequence read to a reference genome, and comprises a plurality of encodings, where each encoding in the plurality of encodings represents a coordinate match status of one or more base positions in the respective sequence read compared with the reference genome.

In some embodiments, each encoding in the plurality of encodings is selected from the group consisting of match “M,” insertion “I,” deletion “D,” skipped “N,” soft-clipping “S,” and hard-clipping “H”.

In some embodiments, each encoding in the plurality of encodings further comprises an indication of a number of base positions in the one or more base positions that have the respective coordinate match status.

In some embodiments, each base position in the plurality of base positions of the target nucleic acid molecule that fails to satisfy a selection criterion is removed from one or more sequence reads in the plurality of sequence reads, and the selection criterion is a measure of central tendency of the number of observations of a respective encoding for the respective base position, across each sequence read in the plurality of sequence reads that align to a region of the reference genome spanning at least the respective base position. In some embodiments, the measure of central tendency is a median or a mode.

In some embodiments, the method further comprises, prior to the obtaining the sequencing dataset, removing from the plurality of sequence reads of the target nucleic acid molecule each base position in the plurality of base positions that satisfies a filtering criterion. In some embodiments, the filtering criterion is satisfied when the plurality of base reads comprises a threshold number of alternative nucleotide base identities at the respective base position. In some embodiments, the filtering criterion is satisfied when, for each respective sequence read in the plurality of sequence reads, the respective base position comprises a positive strand nucleotide base identity of cytosine and a negative strand nucleotide base identity of guanine.

Another aspect of the present disclosure provides a computing system comprising at least one processor and memory storing at least one program to be executed by at least one processor. At least one program comprises instructions for determining a consensus base call for a base position within a plurality of base positions of a target nucleic acid molecule in a biological sample of a subject by any of the methods disclosed herein.

Still another aspect of the present disclosure provides a non-transitory computer readable storage medium storing at least one program for determining a consensus base call for a base position within a plurality of base positions of a target nucleic acid molecule in a biological sample of a subject. At least one program is configured for execution by a computer. At least one program comprises instructions for performing any of the methods disclosed herein.

As disclosed herein, any embodiment disclosed herein when applicable can be applied to any aspect. Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference in their entireties to the same extent as if each such individual publication, patent, and patent application were specifically and individually indicated to be incorporated by reference. In the event of a conflict between a term herein and a term in an incorporated reference, the term herein controls.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 is a block diagram illustrating an example of a computing system in accordance with some embodiments of the present disclosure.

FIGS. 2A and 2B collectively illustrate examples of a flowchart of methods of determining a consensus base call, in accordance with some embodiments of the present disclosure.

FIG. 3 illustrates an example sequence read with multiple possible sequences in the adapter regions, as used in accordance with some embodiments of the present disclosure.

FIG. 4 illustrates an example of bag collapsing, where base reads from multiple sequence reads, each of the same fragment of nucleic acid, are compared in accordance with some embodiments of the present disclosure.

FIG. 5 illustrates an example of building a feature tensor, based on individual base read quality scores and other features that are then transformed into a feature tensor, in accordance with some embodiments of the present disclosure.

FIG. 6 illustrates an example of model weights for predicting a nucleic acid (e.g., making a base call), as performed in accordance with some embodiments of the present disclosure. FIG. 6 includes example weights for predicting nucleotide base A on either a forward strand or a reverse strand. Similar sets of weights are also calculated for each other nucleotide base type (e.g., G, C, and T or U).

FIG. 7 illustrates recalibration of quality scores, in accordance with some embodiments of the present disclosure.

FIG. 8 illustrates an example of reduced error rates using recalibrated quality scores generated in accordance with some embodiments of the present disclosure.

FIGS. 9A and 9B illustrate examples of reduced error rates based on recalibrated quality scores, in accordance with some embodiments of the present disclosure. FIG. 9A is an example of quality scores determined from sequencing of NA12878 gDNA. FIG. 9B is an example of quality scores determined from sequencing of cfDNA.

FIG. 10 illustrates misclassification rates for trinucleotide sets, where trinucleotide context is informative for determining quality scores in accordance with some embodiments of the present disclosure.

FIG. 11 illustrates that the in house proprietary pipeline (herein referred to as Pecan) exhibits high levels of error rates in determining single nucleotide variants (SNVs), as compared with a collapser model trained in accordance with some embodiments of the present disclosure (e.g., a collapser classification model exhibited error rates at least half that of the Pecan pipeline).

FIG. 12 illustrates that the Pecan pipeline filters out variants with low levels of support.

FIGS. 13A, 13B, and 13C collectively illustrate an example of bagging sequence reads into pileups, in accordance with some embodiments of the present disclosure.

FIGS. 14A, 14B, and 14C collectively illustrate examples of decreased error rates in determining consensus base calls, using unique molecular identifier (UMI)-free bagging of whole genome sequencing data, in accordance with some embodiments of the present disclosure.

FIGS. 15A, 15B, 15C, and 15D collectively illustrate the separation of featurized sequence read bag pileups in a principal component analysis space, in accordance with some embodiments of the present disclosure.

FIG. 16 illustrates the error rate of a machine learning (ML)-based collapser using a methylation sequencing validation dataset, in accordance with some embodiments of the present disclosure.

FIG. 17 illustrates the error rates in trinucleotide sets, for a machine learning (ML)-based collapser using a methylation sequencing validation dataset, in accordance with some embodiments of the present disclosure.

FIGS. 18A and 18B collectively illustrate the error rate of a machine learning (ML)-based collapser using a methylation sequencing validation dataset as a function of phred score, in accordance with some embodiments of the present disclosure. FIG. 18A illustrates error rate across all bases. FIG. 18B illustrates error rates for each individual base.

FIG. 19 illustrates the misclassification rates of a machine learning (ML)-based collapser using a methylation sequencing validation dataset as a function of symmetric distance of a base to the end of a nucleic acid fragment, in accordance with some embodiments of the present disclosure.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments. The implementations described herein provide various technical solutions for training a classifier to determine consensus base calls for one or more base positions of a nucleic acid.

Benefits.

An essential component of using DNA sequencing information to diagnose and treat cancer is obtaining high quality sequencing data that enables detection of mutations. Many sequencing methods assign quality scores to base reads (e.g., phred scores) indicating the likelihood of mistaken sequencing (e.g., the likelihood of a mistaken base call being made). See e.g., Brockman et al., 2008, Genome Res 187, 763-770 and Ledergerber et al., 2011, Briefings in Bioinformatics 12(5), 489-497. In typical sequencing methods, multiple base reads are condensed into a base call for an individual base position (e.g., as part of a genome sequence) with a corresponding confidence score in the base call. However, there are drawbacks with applying such a sequencing pipeline to cell-free DNA (cfDNA) sequencing.

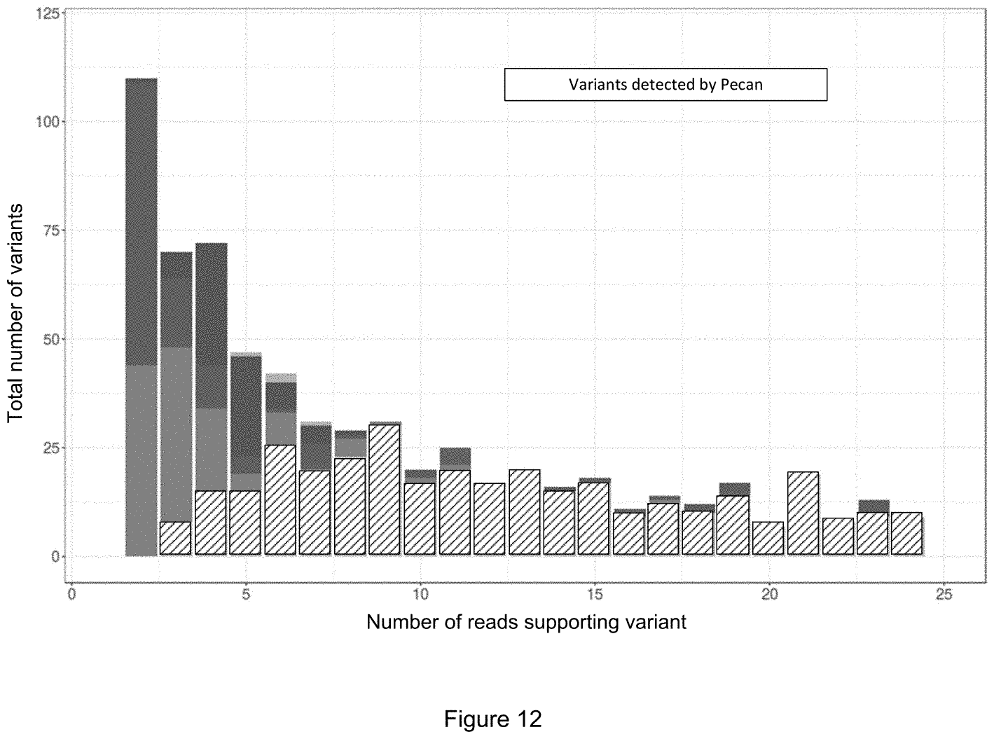

In particular, cfDNA sequencing (which is used particularly for diagnosing cancer) is inherently limited in the amount of original fragments. See e.g., Snyder et al., 2016 Cell 164, 57-68. Traditional methods of determining consensus base calls can use base reads with relatively low quality scores to obtain a base call with a relatively high confidence score simply because of the availability of many independent base reads for a particular base position. However, in instances where there are a limited number of base reads (e.g., cfDNA sequencing) then true variants can be missed. For example, in FIG. 12 described in Example 3, that the in house proprietary variant calling pipeline (“Pecan”) missed 41% of true variants. Similar, conventional alignment tools are disclosed in Schmitt et al., 2012, Proceedings of the National Academy of Sciences 109 (36) 14508-14513; and Schmitt et al., 2014, “Detecting ultralow-frequency mutations by Duplex Sequencing,” Nat Protoc 9, 2586-2606, each of which is hereby incorporated by reference. Sequencing accuracy is especially important for cancer diagnosis, e.g., early stage cancer, where there is often a low level of signal for specific cancer types. See e.g., Aravanis et al., 2017 Cell 168, 571-574 describing the TCGA and CCGA studies (which are described in Examples 1 and 2).

Furthermore, methods such as Pecan can result in erroneous quality scores for base reads, due to the use of hard-coded rules based on cut-off thresholds for duplex/non-duplex reads, stitched/non-stitched reads, read tier and/or base consistency within bags. These hard cut-offs result in loss of information on bag depth and bag consistency when calculating quality scores, preventing the use of the lost information in downstream diagnostic and classification pipelines.

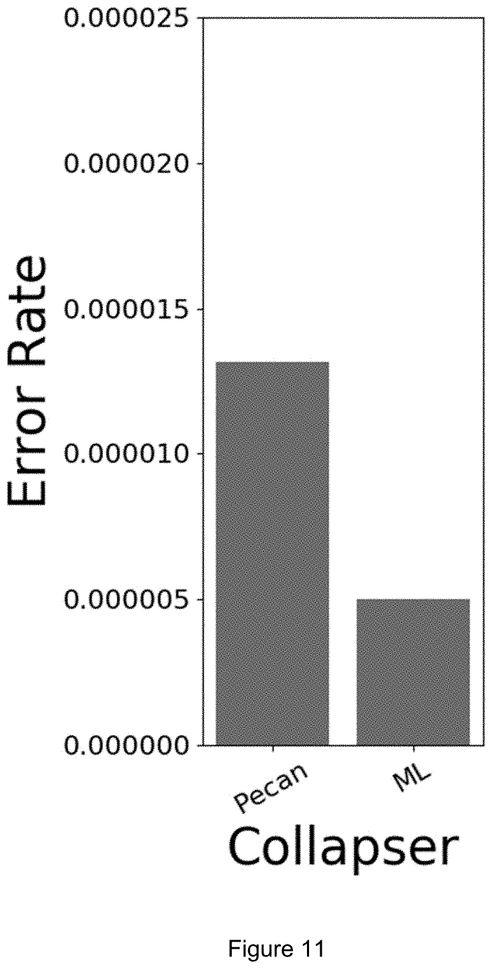

The methods disclosed herein permit accurate nucleotide base calling with a relatively low number of sequence reads, which is of particular benefit for cfDNA sequencing. This is achieved by, for example, calculating calibrated base quality scores. Thus, even though a limited number of base reads are available for any one base position, the calibrated quality scores enable base calling with high confidence scores. These calibrated quality scores take into consideration more data points for each base read than are incorporated in typical base read quality scores, such as phred scores. Using the methods described herein for determining consensus base calls provides improved quality scores for each such consensus base call over previous sequencing methods, as seen in Example 3 with the comparison between the in house proprietary variant calling pipeline (“Pecan”) and a classifier trained as described herein. For example, as shown in FIG. 11, the error rate at the highest quality positions (e.g., the percentage of incorrect base calls) of a method trained as disclosed herein is at least half the error rate of the Pecan method (e.g., in analysis of the same data set with both methods).

Specifically, by avoiding ad hoc, hard-coded decision rules used in conventional techniques, the methods described herein incorporate a greater amount of information into the calculation of base read quality scores. This approach provides advantages over conventional methods in that it is more data-driven and adaptable to assay-specific errors, while further providing a streamlined process for consensus base calling by eliminating read tiers. The model can be trained directly on sequencing data (e.g., cfDNA sequencing data), resulting in quality scores (e.g., phred scores) that are realistic and reflect empirical error rates. Calibration of consensus base quality scores can also improve the output of downstream applications, including, among others, more reliable downstream SNV calling.

Thus, by taking advantage of more of the data available from sequencing results, the methods disclosed herein provide for improved variant calling in downstream sequencing analysis (e.g., due to the higher confidence of individual base calls using recalibrated quality scores), which in turn permits higher confidence in diagnosing diseases with genetic components (e.g., cancer).

Exemplary Method Embodiments.

For example, in some preferred embodiments of the present disclosure, consensus base calls in nucleic acid sequencing are determined by grouping or aggregating (e.g., “bagging”) a plurality of sequence reads corresponding to an original nucleic acid fragment, and obtaining, from a classifier, a consensus base probability for each respective base position in the bag, generated from an input tensor of featurized characteristics of each base read, in the plurality of sequence reads, corresponding to the respective base position (e.g., “collapsing”).

In accordance with some preferred embodiments of the present disclosure, the method comprises obtaining a sequencing dataset comprising a plurality of sequence reads. The sequencing dataset can be, for example, a targeted sequencing dataset and/or a methylation sequencing dataset. Each sequence read in the plurality of sequence reads that corresponds to an original nucleic acid molecule (e.g., that was derived from a target nucleic acid molecule via one or more sequencing reactions) is bagged. The plurality of sequence reads in a bag is interchangeably referred to herein as a “bag pileup” or a “pileup” of sequence reads. For each bag corresponding to a target nucleic acid molecule, the pileup of sequence reads can be generated using duplicate marking, based on the unique molecular identifier, the genomic position of each sequence read in the plurality of sequence reads, and/or visual inspection of optical duplicates in a sequencing flow cell.

A plurality of base positions is represented in the pileup of sequence reads, where each sequence read in the pileup of sequence reads comprises one or more base positions in the plurality of base positions. The plurality of base positions can be filtered, for example, to remove variant positions (e.g., base positions having 3 or more alternate base identities in the pileup) and/or possible methylation positions (e.g., base positions having a cytosine on the forward strand and guanine on the reverse strand) from each sequence read in the pileup.

Collapsing is performed by featurizing the pileup of sequence reads in each bag, at each base position represented in the bag. For example, for each sequence read at a respective base position, features can comprise the base identity (e.g., A, C, G, and T or U), a quality score (e.g., a phred score and/or a mapping quality score), a strand identifier (e.g., a forward strand or a reverse strand of the target nucleic acid molecule), a distance of the base read from the end of the sequence read, a trinucleotide context, a read direction identifier (e.g., a left read or a right read), and/or any other features of a base read desired for training or using a classifier.

For each base position, features are aggregated across the pileup of sequence reads by calculating the adjusted count of each base read comprising the respective features. For example, in some preferred embodiments, a phred score feature is extracted from a sequence read bag by calculating a sum of discounted values for each base read, in the plurality of base reads at the respective base position, having the respective phred score. This discounted value is separately calculated for each base identity (e.g., for base reads of A, C, G, T, and/or U). Each extracted feature is populated into a feature matrix, which is subsequently transformed by flattening into a featurized tensor that can be used as input into a classifier, such as a multinomial logistic regression classifier.

In some embodiments, the classifier is a multivariate logistic regression model, a neural network, a convolutional neural network (deep learning algorithm), a support vector machine (SVM), a Naïve Bayes algorithm, a nearest neighbor algorithm, a random forest algorithm, a decision tree algorithm, a boosted tree algorithm, a regression algorithm, a logistic regression algorithm, a multi-category logistic regression algorithm, a linear discriminant analysis algorithm, or a supervised clustering model.

The input tensor can be used to train an untrained classifier and/or use a trained classifier to predict a nucleotide base identity. Thus, the output from the classifier is a probability or a confidence score for each base identity (e.g., A, C, G, T, and/or U) at the respective base position in the bag. A quality score for each base identity can be further calculated based on the output probability generated by the classifier. In some embodiments, the quality score is calibrated for higher accuracy by binning model-derived quality scores, computing the corresponding error rates based on empirical error, and linearly interpolating the remaining quality scores between estimates.

Finally, consensus sequences can be generated using a plurality of consensus base calls determined by the classifier across the plurality of base positions represented in the bag of sequence reads. Such consensus sequences can be used for downstream applications including SNV calling and/or the detection and diagnosis of human genetic diseases.

Definitions.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within “2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

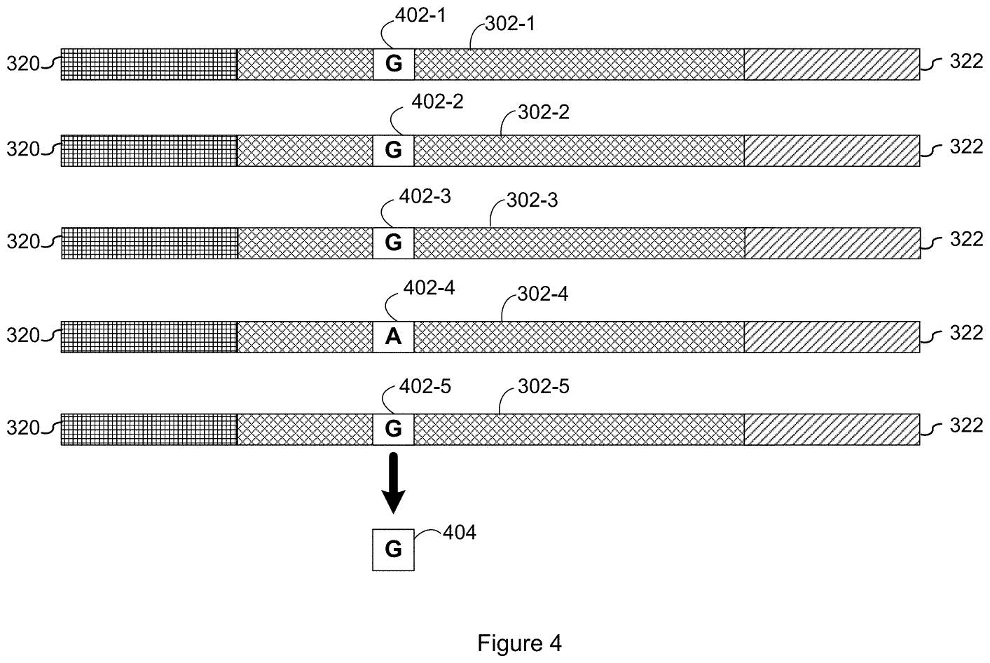

As used herein, the terms “bag,” “bagged,” “bagged read,” or “bagging” refer to a set of sequence reads that either a) have the same UMI (e.g., unique molecular identifier) and/or b) align to the same region of the genome. This is illustrated in FIG. 4, where sequence reads 302-1 to 302-5 are included in a bag (e.g., are bagged) by dint of their shared UMIs (320 and 322). It is not necessary that bagged reads share more than one UMI. In some instances, for example in sequencing methods that do not include tagging of sequence fragments (e.g., pieces of nucleic acid collected from a biological sample), sequence reads do not include UMIs and are instead bagged by genomic location along (e.g., by aligning sequences). Sets of sequence reads in a bag can also be referred to as a “bag pileup.”

As used herein, the term “biological sample,” “patient sample,” or “sample” refers to any sample taken from a subject, which can reflect a biological state associated with the subject, and that includes cell free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. In some embodiments, the biological sample consists of blood, whole blood, plasma, serum, urine, cerebrospinal fluid, fecal, saliva, sweat, tears, pleural fluid, pericardial fluid, or peritoneal fluid of the subject. In such embodiments, the biological sample is limited to blood, whole blood, plasma, serum, urine, cerebrospinal fluid, fecal, saliva, sweat, tears, pleural fluid, pericardial fluid, or peritoneal fluid of the subject and does not contain other components (e.g., solid tissues, etc.) of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis. A biological sample can be obtained from a subject invasively (e.g., surgical means) or non-invasively (e.g., a blood draw, a swab, or collection of a discharged sample).

As used herein, the term “cancer” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion, and metastasis. A “benign” tumor can be well differentiated, have characteristically slower growth than a malignant tumor and remain localized to the site of origin. In addition, in some cases a benign tumor does not have the capacity to infiltrate, invade, or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites.

As used herein, the terms “cell-free nucleic acid,” “cell-free DNA,” and “cfDNA” interchangeably refer to nucleic acid fragments that are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject (e.g., bloodstream). Cell-free nucleic acids are interchangeably referred to herein as “circulating nucleic acids.” Examples of the cell-free nucleic acids include but are not limited to RNA, mitochondrial DNA, or genomic DNA. Cell-free nucleic acids can originate from one or more healthy cells and/or from one or more cancer cells.

As used herein, the term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “CpG site” refers to a region of a DNA molecule where a cytosine nucleotide is followed by a guanine nucleotide in the linear sequence of bases along its 5′ to 3′ direction. “CpG” is a shorthand for 5′-C-phosphate-G-3′ that is cytosine and guanine separated by only one phosphate group; phosphate links any two nucleotides together in DNA. Cytosines in CpG dinucleotides can be methylated to form 5-methylcytosine.

As used herein, the term “fragment” is used interchangeably with “nucleic acid fragment” (e.g., a DNA fragment) and “cell-free nucleic acid molecule,” and refers to a portion of a polynucleotide or polypeptide sequence that comprises at least three consecutive nucleotides. In the context of sequencing of nucleic cell-free nucleic acid fragments found in a biological sample, the terms “fragment” and “nucleic acid fragment” interchangeably refer to a cell-free nucleic acid molecule that is found in the biological sample. In such a context, the sequencing (e.g., whole genome sequencing, targeted sequencing, etc.) forms one or more copies of all or a portion of such a nucleic acid fragment in the form of one or more corresponding sequence reads. Such sequence reads, which in fact may be PCR duplicates of the original nucleic acid fragment, therefore “represent” or “support” the nucleic acid fragment. There may be a plurality of sequence reads that each represent or support a particular nucleic acid fragment in the biological sample (e.g., PCR duplicates). In some embodiments, nucleic acid fragments are cell-free nucleic acids.

As used herein, the phrase “healthy” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “level of cancer” refers to whether cancer exists (e.g., presence or absence), a stage of a cancer, a size of tumor, presence or absence of metastasis, the total tumor burden of the body, and/or other measure of a severity of a cancer (e.g., recurrence of cancer). The level of cancer can be a number or other indicia, such as symbols, alphabet letters, and colors. The level can be zero. The level of cancer can also include premalignant or precancerous conditions (states) associated with mutations or a number of mutations. The level of cancer can be used in various ways. For example, screening can check if cancer is present in someone who is not known previously to have cancer. Assessment can investigate someone who has been diagnosed with cancer to monitor the progress of cancer over time, study the effectiveness of therapies or to determine the prognosis. In some embodiments, the prognosis can be expressed as the chance of a subject dying of cancer, or the chance of the cancer progressing after a specific duration or time, or the chance of cancer metastasizing. Detection can comprise ‘screening’ or can comprise checking if someone, with suggestive features of cancer (e.g., symptoms or other positive tests), has cancer. A “level of pathology” can refer to level of pathology associated with a pathogen, where the level can be as described above for cancer. When the cancer is associated with a pathogen, a level of cancer can be a type of a level of pathology.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous cfDNA methylation can identified as hypermethylation or hypomethylation, both of which may be indicative of cancer status. As is well known in the art, DNA methylation anomalies (compared to healthy controls) can cause different effects, which may contribute to cancer. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts (e.g., 5′-CHG-3′ and 5′-CHH-3′) where H is adenine, cytosine, or thymine. Cytosine methylation can also be in the form of 5-hydroxymethylcytosine. Methylation of DNA can include methylation of non-cytosine nucleotides, such as N6-methyladenine. For example, methylation data (e.g., density, distribution, pattern, or level of methylation) from different genomic regions can be converted to one or more vector set and analyzed by methods and systems disclosed herein.

Various challenges arise in the identification of anomalously methylated cfDNA fragments. First, determining a subject's cfDNA to be anomalously methylated only holds weight in comparison with a group of control subjects, such that if the control group is small in number, the determination loses confidence with the small control group. Additionally, among a group of control subjects' methylation status can vary which can be difficult to account for when determining a subject's cfDNA to be anomalously methylated. On another note, methylation of a cytosine at a CpG site causally influences methylation at a subsequent CpG site.

Those of skill in the art will appreciate that the principles described herein are equally applicable for the detection of methylation in a non-CpG context, including non-cytosine methylation. Further, methylation state vectors may contain elements that are generally vectors of sites where methylation has or has not occurred (even if those sites are not CpG sites specifically). With that substitution, the remainder of the processes described herein are the same, and consequently the inventive concepts described herein are applicable to those other forms of methylation.

As used herein, the term “misclassified example” refers to an example base call comprising a false positive (e.g., a base call mistakenly indicating a mutation). False positive base calls are frequently referred to as “sequencing error.” Such misclassified examples can be used to determine which sequence read features are more likely to be associated with false positive base calls (e.g., by observing where misclassified examples cluster in principal component analysis). In some embodiments, a misclassified example refers to a base call comprising a false negative (e.g., a base calls that mistakenly does not indicate a mutation). In some embodiments, a set of misclassified examples comprises both false negatives and false positives. In some embodiments, a set of misclassified examples have low bag depth (e.g., fall below a predefined threshold depth). In some embodiments, a set of misclassified examples are not clustered (e.g., with regard to bag depth). In some embodiments, a set of misclassified examples have high bag depth. In some embodiments, the high bag depth of misclassified examples is due to DNA damage or PCR error at an early stage of amplification. In some embodiments, a set of misclassified examples are tightly clustered (e.g., in terms of bag depth).

As used herein, the term “methylation state vector” or “methylation status vector” refers to a vector comprising multiple elements, where each element indicates methylation status of a methylation site in a DNA molecule comprising multiple methylation sites, in the order they appear from 5′ to 3′ in the DNA molecule. For example, <Mx, Mx+J, Mx+2>, <Mx, Mx+1, Ux+2>, . . . , <Ux, Ux+1, Ux+2> can be methylation vectors for DNA molecules comprising three methylation sites, where M represents a methylation site that is in a methylated state and U represents a methylation site in an unmethylated state. U.S. Patent Application No. 62/948,129, entitled “Cancer Classification Using Patch Convolutional Neural Networks,” filed Dec. 13, 2019, which is hereby incorporated by reference in its entirety, further discloses methods of determining methylation state vectors. For example, for each sequence read in a plurality of sequence reads obtained from a biological sample of a subject, a respective location and respective methylation state is determined for each of one or more CpG cites based on alignment to a reference genome (e.g., the reference genome of the subject). A respective methylation state vector is determined for each fragment, where the respective methylation state vector is associated with a location of the fragment in the reference genome (e.g., as specified by the position of the first CpG site in each fragment, or another similar metric) and comprises a number of CpG sites in the fragment, as well as the methylation state of each CpG site in the fragment whether methylated (e.g., denoted as M), unmethylated (e.g., denoted as U), or indeterminate (e.g., denoted as I). Observed states are states of methylated and unmethylated; whereas, an unobserved state is indeterminate.

As used herein, the term “mutation” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. The term “variant” refers to a region of the genome that differs between individuals of the same species (e.g., a region of the genome that comprises one or more mutations). A region of the genome corresponding to a variant may be mutated in multiple ways at a single location (e.g., a single nucleotide may be converted to an ‘A’ or to a ‘G’) or may be mutated at multiple locations. The term “allele” refers to one of two or more forms of a gene, where each form includes a mutation. An allele may correspond, for example, to a single nucleotide polymorphism (SNP), where a single base is mutated. Each allele is a variant of a gene. Each variant may comprises more than one allele. A mutation in the sequence (e.g., in one or more genes) of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

The term “normalize” as used herein means transforming a value or a set of values to a common frame of reference for comparison purposes. For example, when a diagnostic ctDNA level is “normalized” with a baseline ctDNA level, the diagnostic ctDNA level is compared to the baseline ctDNA level so that the amount by which the diagnostic ctDNA level differs from the baseline ctDNA level can be determined.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), DNA hybrids, and polyamide nucleic acids (PNAs), all of which can be in single- or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments, nucleic acids comprise nucleosomes, fragments or parts of nucleosomes or nucleosome-like structures. Nucleic acids sometimes comprise protein (e.g., histones, DNA binding proteins, and the like). Nucleic acids analyzed by processes described herein sometimes are substantially isolated and are not substantially associated with protein or other molecules. Nucleic acids also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides include deoxyadenosine, deoxycytidine, deoxyguanosine and deoxythymidine. A derived nucleic acid may be prepared using a nucleic acid obtained from a subject as a template. Each nucleic acid molecule has a 3′ and a 5′ end. The 3′ (e.g., three prime) end refers to an end of a nucleic acid molecule where the sugar phosphate backbone of the nucleic acid molecule terminates at a hydroxyl group on the third carbon of a furanose molecule. The 5′ (e.g., five prime) end refers to an end of a nucleic acid molecule where the sugar phosphate backbone of the nucleic acid molecule terminates at phosphate group on the fifth carbon of a furanose molecule. The term “upstream” used with regard to a DNA sequence refers to a relative position in a reference genome that is towards the 5′ end of a sequence read. The term “downstream” used with regard to a DNA sequence refers to a relative position in a reference genome that is towards the 3′ end of a sequence read.

As used herein, the term “reference genome” refers to any particular known, sequenced, or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As disclosed herein, the term “regions of a reference genome,” “genomic region,” or “chromosomal region” refers to any portion of a reference genome, contiguous or non-contiguous. It can also be referred to, for example, as a bin, a partition, a genomic portion, a portion of a reference genome, a portion of a chromosome and the like. In some embodiments, a genomic section is based on a particular length of genomic sequence. In some embodiments, a method can include analysis of multiple mapped sequence reads to a plurality of genomic regions. Genomic regions can be approximately the same length or the genomic sections can be different lengths. In some embodiments, genomic regions are of about equal length. In some embodiments, genomic regions of different lengths are adjusted or weighted. In some embodiments, a genomic region is about 10 kilobases (kb) to about 500 kb, about 20 kb to about 400 kb, about 30 kb to about 300 kb, about 40 kb to about 200 kb, and sometimes about 50 kb to about 100 kb. In some embodiments, a genomic region is about 100 kb to about 200 kb. A genomic region is not limited to contiguous runs of sequence. Thus, genomic regions can be made up of contiguous and/or non-contiguous sequences. A genomic region is not limited to a single chromosome. In some embodiments, a genomic region includes all or part of one chromosome, or all or part of two or more chromosomes. In some embodiments, genomic regions may span one, two, or more entire chromosomes. In addition, the genomic regions may span joint or disjointed portions of multiple chromosomes.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median, or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median, or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing breadth” refers to what fraction of a particular reference genome (e.g., human reference genome) or part of the genome has been analyzed. The denominator of the fraction can be a repeat-masked genome, and thus 100% can correspond to all of the reference genome minus the masked parts. A repeat-masked genome can refer to a genome in which sequence repeats are masked (e.g., sequence reads align to unmasked portions of the genome). Any parts of a genome can be masked, and thus one can focus on any particular part of a reference genome. Broad sequencing can refer to sequencing and analyzing at least 0.1% of the genome.

As used herein, the terms “sequencing depth,” “coverage,” and “coverage rate” are used interchangeably herein to refer to the number of times a locus is covered by a consensus sequence read corresponding to a unique nucleic acid target molecule (e.g., “nucleic acid fragment”) aligned to the locus; e.g., the sequencing depth is equal to the number of unique nucleic acid target fragments (e.g., excluding PCR sequencing duplicates) covering the locus. The locus can be as small as a nucleotide, or as large as a chromosome arm, or as large as an entire genome. Sequencing depth can be expressed as “YX”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a loci or a haploid genome, or a whole genome, respectively, is sequenced. When a mean depth is quoted, the actual depth for different loci included in the dataset can span over a range of values. The term “bag depth” refers to the sequencing depth for a particular genomic locus. Ultra-deep sequencing can refer to at least 100× in sequencing depth (e.g., bag depth) at a locus.

As used herein, the term “single nucleotide variant” or “SNV” refers to a substitution of one nucleotide at a position (e.g., site) of a nucleotide sequence, e.g., a sequence corresponding to a target nucleic acid molecule from an individual, to a nucleotide that is different from the nucleotide at the corresponding position in a reference genome. A substitution from a first nucleobase X to a second nucleobase Y may be denoted as “X>Y.” For example, a cytosine to thymine SNV may be denoted as “C>T.” In some embodiments, a SNV does not result in a change in amino acid expression (a synonymous variant). In some embodiments, a SNV results in a change in amino acid expression (a non-synonymous variant).

As used herein, the terms “size profile” and “size distribution” can relate to the sizes of DNA fragments in a biological sample. A size profile can be a histogram that provides a distribution of an amount of DNA fragments at a variety of sizes. Various statistical parameters (also referred to as size parameters or just parameter) can distinguish one size profile to another. One parameter can be the percentage of DNA fragment of a particular size or range of sizes relative to all DNA fragments or relative to DNA fragments of another size or range.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any stage (e.g., a man, a woman, or a child).

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother versus fetus) or to healthy cells versus tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “vector” is an enumerated list of elements, such as an array of elements, where each element has an assigned meaning. As such, the term “vector” as used in the present disclosure is interchangeable with the term “tensor.” As an example, if a vector comprises base read features for 10,000 base reads, there exists a predetermined element in the vector for each one of the 10,000 base reads. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents a first feature of base read 1 of a plurality of base reads, that element 2 represents a second feature of base read 1 of a plurality of base reads, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Exemplary System Embodiments.

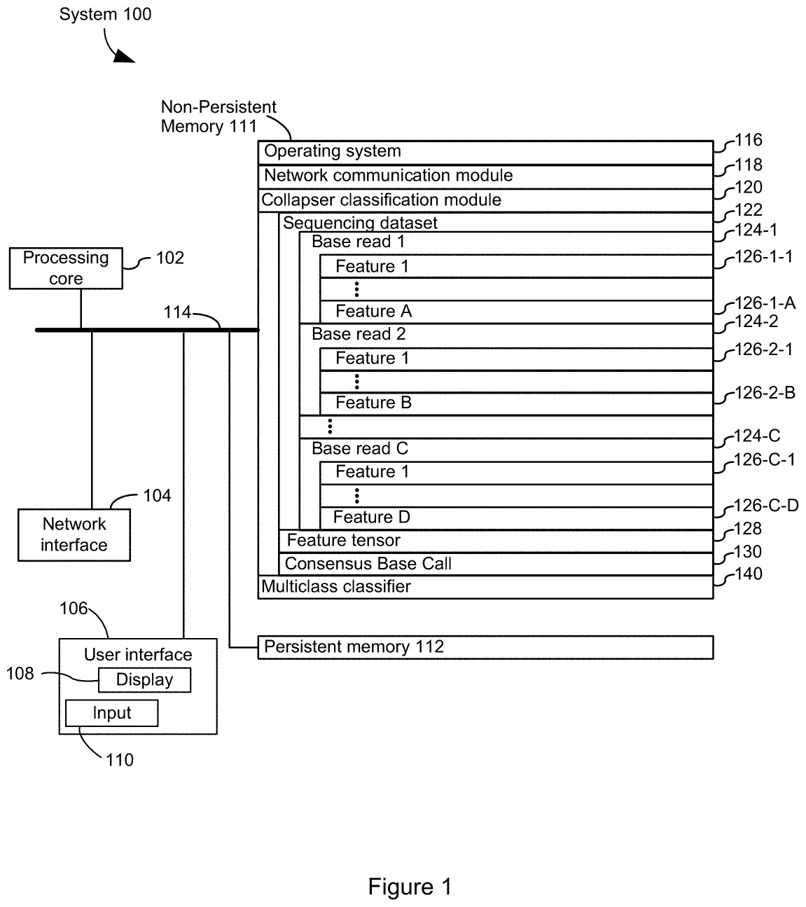

Details of an exemplary system are now described in conjunction with FIG. 1. FIG. 1 is a block diagram illustrating a system 100 in accordance with some implementations. The system 100 in some implementations includes at least one or more processing units CPU(s) 102 (also referred to as processors), one or more network interfaces 104, a display 106 having a user interface 108, an input device 110, a memory 111, and one or more communication buses 114 for interconnecting these components. The one or more communication buses 114 optionally include circuitry (sometimes called a chipset) that interconnects and controls communications between system components.

The memory 111 may be a non-persistent memory, a persistent memory, or any combination thereof. Non-persistent memory typically includes high-speed random access memory, such as DRAM, SRAM, DDR RAM, ROM, EEPROM, flash memory, whereas the persistent memory typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. Persistent memory 112 optionally includes one or more storage devices remotely located from the CPU(s) 102. The persistent memory 112, and the non-volatile memory device(s) within the non-persistent memory 112, comprise non-transitory computer readable storage medium. Regardless of its specific implementation, the memory 111 comprises at least one non-transitory computerreadable storage medium, and it stores thereon computer-executable executable instructions which can be in the form of programs, modules, and data structures.

In some embodiments, as shown in FIG. 1, the memory 111 stores the following programs, modules, and data structures, or a subset thereof:

an operating system 116, which includes procedures for handling various basic system services and for performing hardware-dependent tasks;

an optional network communication module (or instructions) 118 for connecting the system 100 with other devices and/or to a communication network;

a collapse classification module 120 for determining consensus base calls from sequencing datasets;

a sequencing dataset 122 comprising, for a subject, a plurality of base reads (124-1, . . . 124-C) for a first base position, where each base read includes two or more features (e.g., 126-1-1, . . . 126-1-A);

a transformed feature tensor 128 that represents a distribution of the at least two features associated with each base read in the plurality of base reads and is derived from the sequencing dataset 122;

a consensus base call 130 comprising a predicted nucleotide base that is determined by assessing the features tensor 128; and

a classifier 140 that is used to assess the feature tensor 128 and determine a consensus base call (e.g., for each base position in the plurality of base positions).

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the memory 111 optionally stores a subset of the modules and data structures identified above. Furthermore, in some embodiments, the memory stores additional modules and data structures not described above. In some embodiments, one or more of the above-identified elements is stored in a computer system other than that of visualization system 100, that is addressable by the visualization system 100 so that the visualization system 100 may retrieve all or a portion of such data when needed.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items can be separate. Moreover, although FIG. 1 depicts certain data and modules in the memory 111 (which can be non-persistent or persistent memory), it should be appreciated that these data and modules, or portion(s) thereof, may be stored in more than one memory. For example, in some embodiments, at least the sequencing dataset 122, the feature tensor 128, and the classifier 140 are stored in a remote storage device that can be a part of a cloud-based infrastructure. In some embodiments, at least the sequencing dataset 122 is stored on a cloud-based infrastructure. In some embodiments, the feature tensor 128 and the classifier 140 can also be stored in the remote storage device(s).

Specific Embodiments of the Disclosure.

While a system in accordance with the present disclosure has been disclosed with reference to FIG. 1, methods in accordance with the present disclosure are now detailed. Any of the methods in accordance with embodiments of the present disclosure can make use of any of the assays, algorithms, or techniques, or combinations thereof, disclosed in U.S. Patent Publication No. US20180237863 and/or International Patent Publication No. WO2018081130, each of which is hereby incorporated herein by reference in its entirety, in order to determine a cancer condition in a test subject or a likelihood that the subject has the cancer condition.

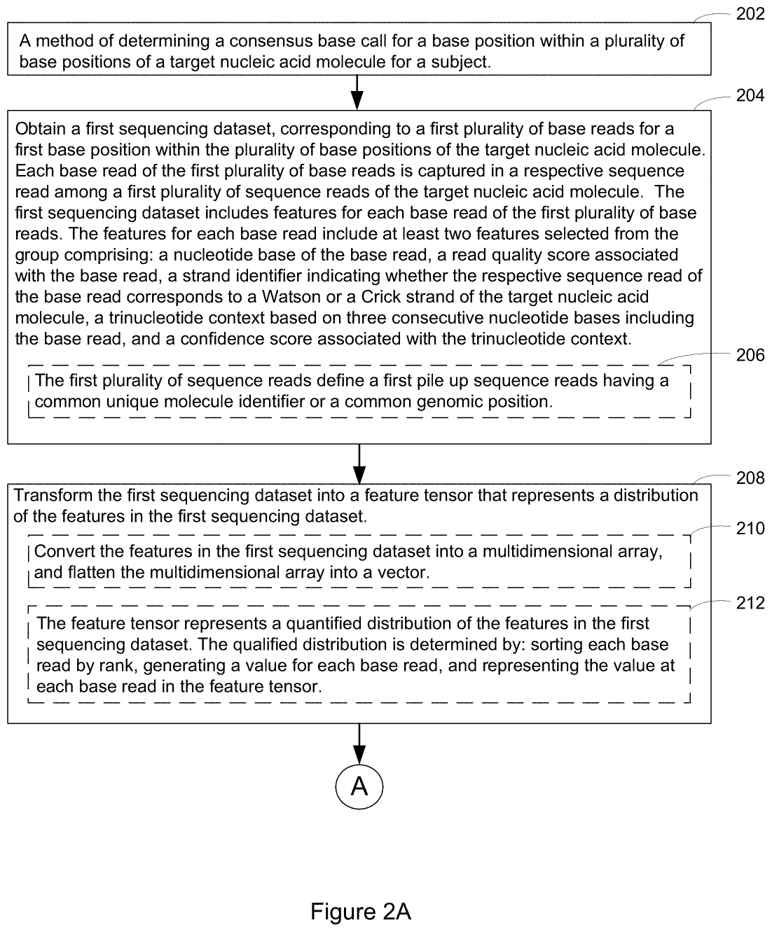

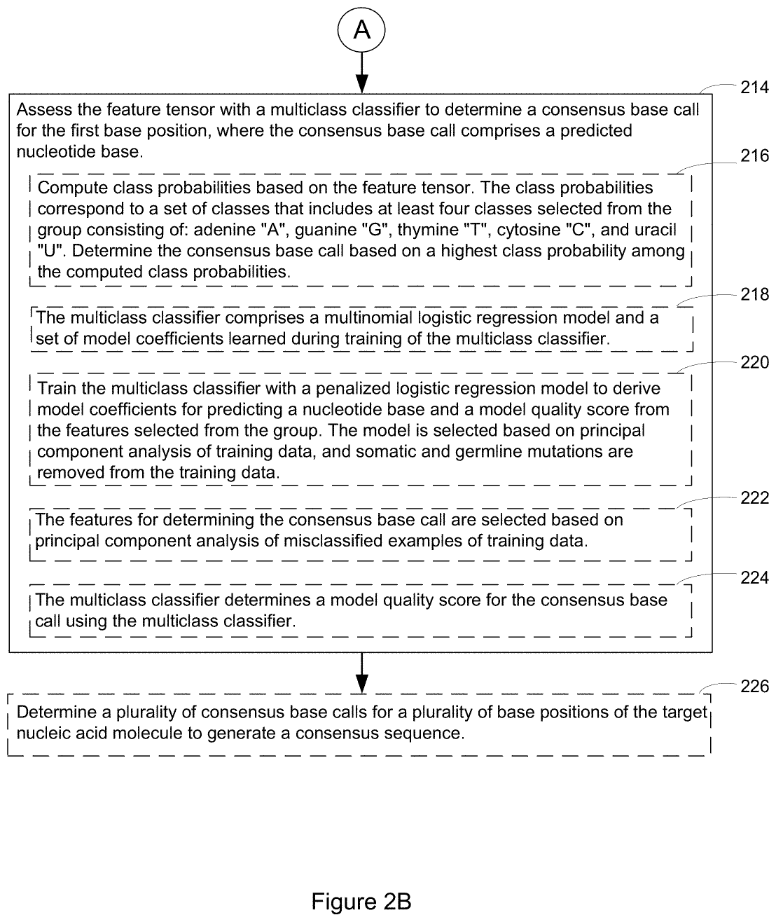

FIG. 2 illustrates an overview of the techniques in accordance with some embodiments of the present disclosure. In the described embodiments, various methods of collapsing nucleic acid base reads into base call are described. In some embodiments, the various methods are encoded in collapse classification module 120.

Block 202. Referring to block 202 of FIG. 2A, the method determines a consensus base call for a base position within a plurality of base positions of a target nucleic acid molecule for a subject. In some embodiments, the target nucleic acid molecule comprises cell-free nucleic acid obtained from a biological sample of the subject.

In some embodiments, the subject and/or the biological sample are any of the examples defined above (see, Definitions).

In some embodiments, the subject has a cancer condition. In some embodiments, the cancer condition is a presence or absence of cancer, a type of a cancer, a stage of a cancer, and/or a tissue-of-origin. In some embodiments, the biological sample is a matched sample (e.g., the biological sample is a cancer sample that has a corresponding non-cancer reference sample).

Sample Processing.

In some embodiments, the biological sample is processed to extract cell-free nucleic acids in preparation for sequencing analysis. By way of a non-limiting example, in some embodiments, cell-free nucleic acid is extracted from a blood sample collected from a subject in K2 EDTA tubes. Samples are processed within two hours of collection by double spinning of the blood first at ten minutes at 1000 g then plasma ten minutes at 2000 g. The plasma is then stored in 1 ml aliquots at −80° C. In this way, a suitable amount of plasma (e.g., 1-5 ml) is prepared from the first and/or second biological sample for the purposes of cell-free nucleic acid extraction. In some such embodiments, cell-free nucleic acid is extracted using the QIAamp Circulating Nucleic Acid kit (Qiagen) and eluted into DNA Suspension Buffer (Sigma). In some embodiments, the purified cell-free nucleic acid is stored at −20° C. until use. See, for example, Swanton et al., 2017, “Phylogenetic ctDNA analysis depicts early stage lung cancer evolution,” Nature, 545(7655): 446-451, which is hereby incorporated herein by reference in its entirety. Other equivalent methods can be used to prepare cell-free nucleic acid using biological methods for the purpose of sequencing, and all such methods are within the scope of the present disclosure.

In some embodiments, the cell-free nucleic acid that is obtained from the biological sample is in any form of nucleic acid, or a combination thereof. For example, in some embodiments, the cell-free nucleic acid that is obtained from a biological sample is a mixture of RNA and DNA. In some embodiments, each respective nucleic acid molecule in the cell-free nucleic acids that are obtained from the biological sample of the subject is DNA. In some embodiments, the cell-free nucleic acids of the first and second biological samples have undergone a conversion treatment comprising converting unmethylated cytosines or converting methylated cytosines. In some embodiments, the conversion treatment comprises conversion of one or more unmethylated cytosines or one or more methylated cytosines to a corresponding one or more uracils. In some such embodiments, the conversion of one or more unmethylated cytosines or one or more methylated cytosines comprises a chemical conversion, an enzymatic conversion, or combinations thereof.

In some embodiments, the method further comprises obtaining one or more target nucleic acid molecules from a biological sample of the subject and fragmenting the one or more target nucleic acid molecules, thereby obtaining a plurality of nucleic acid fragments. In some such embodiments, the fragmentation is performed via, for example, shearing, sonication, and/or enzymatic or chemical digestion. In some embodiments, a target nucleic acid molecule is a nucleic acid fragment.

In some embodiments, the method further comprises preparing a sequencing library, where the sequencing library comprises a plurality of nucleic acid fragments.

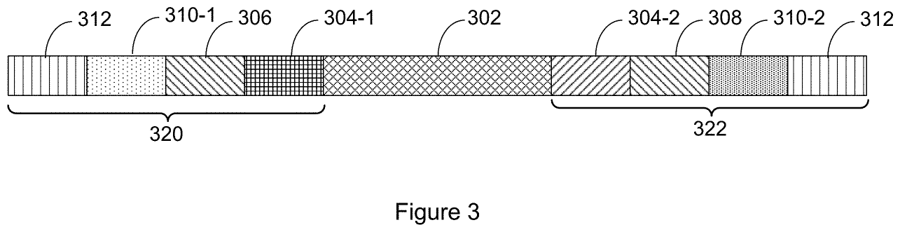

In some embodiments, e.g., preparatory to sequencing nucleic acids derived from the obtained nucleic acids, each derived nucleic acid molecule is ligated to a respective first terminal region (320) and a respective second terminal region (322) in a plurality of terminal regions (e.g., adapters). As illustrated in FIG. 3, each respective terminal region in the plurality of terminal regions comprises a unique sample index (306/308). For example, in some embodiments, the unique sample index identifies the biological sample from which the nucleic acid molecule was obtained. Thus, each respective sequence read (302) in a plurality of sequence reads derived from the obtained cell-free nucleic acids is uniquely identifiable.

In some embodiments, each respective obtained nucleic acid molecule is DNA that comprises a forward strand and a reverse strand, where each of the forward and reverse strands is ligated to a different first and second terminal region in the plurality of terminal regions. In some embodiments, each terminal region in the plurality of terminal regions includes a sequencing primer site (e.g., 304-1 and 304-2).

For example, in some embodiments, the obtained nucleic acid molecule is a double stranded DNA molecule that is ligated to two different double stranded adapters (e.g., a first adapter and a second adapter), where each adapter is ligated to a different end of the double stranded DNA molecule. In some such embodiments, each strand of each adapter comprises a different nucleic acid sequence, thereby ligating each of the forward and reverse strands in the double stranded DNA molecule to a different first and second terminal region. Thus, in some such embodiments, the forward strand of the double stranded DNA molecule comprises a unique first terminal region and a unique second terminal region, and the reverse strand of the double stranded DNA molecule comprises a unique third terminal region and a unique fourth terminal region, where the first, second, third, and fourth terminal regions each comprise a different nucleic acid sequence. In some such embodiments, each terminal region in the first, second, third, and fourth terminal regions includes a sequencing primer site, where each sequencing primer site is unique to the respective terminal region (e.g., the first, second, third and fourth terminal regions each comprise a respective first, second, third, and fourth sequencing primer site, such that no two sequencing primer sites are the same. In some alternative embodiments, the first and third terminal regions each comprise a first sequencing primer site, and the second and fourth terminal regions each comprise a second sequencing primer site, where the first sequencing primer site is different from the second sequencing primer site.

In some embodiments, each sequencing primer site in each respective terminal region in the plurality of terminal regions comprises a binding sequence that is complementary to a sequencing primer. In some such embodiments, two sequencing primer sites that are the same comprise binding sequences that are the same (e.g., both sequencing primer sites will bind to the same sequencing primer), whereas two sequencing primer sites that are different comprise different binding sequences (e.g., each sequencing primer site will bind to a different sequencing primer). Unique sequencing primers complementary to unique sequencing primer sites (e.g., 304-1 and/or 304-2) can be used to distinguish the strand polarity (e.g., forward (+) and reverse (−) strand) and/or the direction of sequencing (e.g., left (R1) and right (R2) sequencing reactions in paired-end sequencing) during downstream analysis of sequence reads.

In some embodiments, each obtained nucleic acid molecule is ligated to a respective first terminal region (320) and a respective second terminal region (322) in a plurality of terminal regions (e.g., adapters) via ligation reaction (PCR) reaction, prior to the obtaining a sequencing dataset. In some embodiments, two different PCR primers are used to amplify a nucleic acid molecule obtained from a biological sample of the subject, where the first PCR primer (e.g., the forward primer) comprises a binding sequence that is complementary to the forward strand and the second PCR primer (e.g., the reverse primer) comprises a binding sequence that is complementary to the reverse strand. In some embodiments, PCR amplification of each nucleic acid molecule further comprises extension of the respective strand by one or more terminal regions (e.g., adapters). In some embodiments, PCR is performed to produce a sequencing library comprising a plurality of amplified nucleic acid fragments.

Sequencing Nucleic Acids.

The sequencing can comprise any form of sequencing that can be used to obtain a number of sequence reads measured from nucleic acids, including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and nanopore sequencing also can be used to obtain sequence reads 140 from the nucleic acid obtained from the biological sample.

In some embodiments, sequencing-by-synthesis and reversible terminator-based sequencing (e.g., Illumina's Genome Analyzer; Genome Analyzer II; HISEQ 2000; HISEQ 2500 (Illumina, San Diego Calif.)) is used to obtain sequence reads from the nucleic acid obtained from a biological sample of a subject in order to form the sequencing dataset 122. In some such embodiments, millions of nucleic acid (e.g., DNA) fragments are sequenced in parallel. In one example of this type of sequencing technology, a flow cell is used that contains an optically transparent slide with eight individual lanes on the surfaces of which are bound oligonucleotide anchors (e.g., adaptor primers). A flow cell often is a solid support that is configured to retain and/or allow the orderly passage of reagent solutions over bound analytes. In some instances, flow cells are planar in shape, optically transparent, generally in the millimeter or sub-millimeter scale, and often have channels or lanes in which the analyte/reagent interaction occurs. In some embodiments, a nucleic acid sample can include a signal or tag that facilitates detection. In some such embodiments, the acquisition of sequence reads from the nucleic acid obtained from a biological sample includes obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

In some embodiments, the sequencing is targeted sequencing. In some alternative embodiments, the sequencing is whole genome sequencing.

In some embodiments, the sequencing is a methylation sequencing (e.g., using bisulfite for conversion). In some such embodiments, the methylation sequencing is whole-genome methylation sequencing or targeted methylation sequencing using a plurality of nucleic acid probes. In some embodiments, a respective probe in the plurality of probes includes a respective nucleic acid sequence that varies with respect to the reference genomic sequence. In some embodiments, the methylation sequencing is whole-genome bisulfite sequencing (WGBS).

In some embodiments, where the sequencing is targeted methylation sequencing, the plurality of probes comprises 1,000 to 2,000,000 probes, where each probe is designed to bind and enrich cell-free nucleic acids in the biological sample that contain at least one predetermined CpG site (e.g., and/or epigenetic feature). In some embodiments, the plurality of probes comprises 1000 probes or more, 2000 probes or more, 3000 probes or more, 4000 probes or more, 5000 probes or more, 6000 probes or more, 7000 probes or more, 8000 probes or more, 9000 probes or more, 10,000 probes or more, 20,000 probes or more, 30,000 probes or more, 40,000 probes or more, 50,000 probes or more, 60,000 probes or more, 70,000 probes or more, 80,000 probes or more, 90,000 probes or more, 100,000 probes or more, 200,000 probes or more, 300,000 probes or more, 400,000 probes or more, 500,000 probes or more, 600,000 probes or more, 700,000 probes or more, 800,000 probes or more, 900,000 probes or more, 1,000,000 probes or more, 1,100,000 probes or more, 1,200,000 probes or more, 1,300,000 probes or more, 1,300,000 probes or more, 1,400,000 probes or more, or 1,500,000 probes or more.

In some embodiments, the methylation sequencing detects one or more 5-methylcytosine (5 mC) and/or 5-hydroxymethylcytosine (5 hmC) in respective fragments. In some embodiments, the methylation sequencing comprises conversion of one or more unmethylated cytosines or one or more methylated cytosines to a corresponding one or more uracils, where the one or more uracils are detected during the methylation sequencing as one or more corresponding thymines.

In some embodiments, the methylation state of a CpG site in the corresponding plurality of CpG sites is methylated when the CpG site is determined by the methylation sequencing to be methylated, and unmethylated when the CpG site is determined by the methylation sequencing to not be methylated. In some embodiments, a methylated state is represented as “M,” and an unmethylated state is represented as “U”. For example, in some embodiments, the methylation state can include but is not limited to: unmethylated, methylated, ambiguous (e.g., meaning the underlying CpG is not covered by any reads in the pair of sequence reads), variant (e.g., meaning that the read is not consistent with a CpG occurring in its expected position based on the reference sequence and can be caused by a real variant at the site or a sequence error), or conflict (e.g., when the two reads both overlap a CpG but are not consistent). See, e.g., U.S. Provisional Patent Application 62/948,129, entitled “Cancer classification using patch convolutional neural networks,” filed Dec. 13, 2019, which is hereby incorporated herein by reference in its entirety.

In some embodiments, the sequencing is paired-end sequencing. In some embodiments, the sequencing is single-end sequencing.

In some embodiments, the biological sample is sequenced using two or more sequencing methods (e.g., a targeted sequencing and a methylation sequencing), thus obtaining matched sequencing datasets. In some embodiments, two biological samples are obtained from a subject, where the first biological sample is sequenced using a first sequencing method, and the second biological sample is sequenced using a second sequencing method that is different from the first sequencing method, thus obtaining matched sequencing datasets.

Sequence Reads.

Blocks 204-206. Referring to block 204, the method obtains a sequencing dataset 122, corresponding to a plurality of base reads 124 for a first base position within the plurality of base positions of a target nucleic acid molecule. Each base read 124 of the plurality of base reads is captured in (e.g., is from) a respective sequence read among a plurality of sequence reads of the target nucleic acid molecule. The first sequencing dataset includes features for each base read of the plurality of base reads.

The features for each base read include two or more features selected from the following features: a nucleotide base of the base read, a read quality score associated with the base read, a strand identifier indicating whether the respective sequence read of the base read corresponds to a forward or a reverse strand of the target nucleic acid molecule, a trinucleotide context based on three consecutive nucleotide bases including the base read, and a confidence score associated with the trinucleotide context.

In some embodiments, the strand identifier feature indicates whether the respective sequence read of the respective base read is from a forward strand or a reverse strand of the target nucleic acid molecule. In some embodiments, this feature is available for sequence reads that are derived from double-stranded target nucleic acid fragments (e.g., this feature is not available when the target molecule is either single-stranded RNA or DNA). In some embodiments, the sequencing read direction indication is binary (e.g., a “1” indicates that the respective sequence read is from a forward strand of the target nucleic acid molecule and a “0” indicates that the respective sequence read is from a reverse strand of the target nucleic acid).

In some embodiments, the trinucleotide context further includes a respective set of trinucleotide discounted values, one trinucleotide discounted value for each of the three base reads comprising the trinucleotide context. In some embodiments, each trinucleotide discounted value includes a respective set of weights that further comprise, for each respective sequencing quality score in a set of sequencing quality scores, for each respective base identity in the set of base identities, for each respective trinucleotide context in a plurality of trinucleotide contexts, a respective weight for the respective base identity for the respective read quality score, for the respective trinucleotide context of the respective base position.

In some embodiments, each respective sequence read in the plurality of sequence reads comprises a copy of all or a portion of the forward strand or the reverse strand of the respective derived nucleic acid molecule flanked by at least the first or second terminal region (e.g., 320 and 322) of the forward strand or the reverse strand of the respective derived nucleic acid molecule. In some embodiments, each respective sequence read in the plurality of sequence reads comprises a copy of all or a portion of the forward strand or the reverse strand of the respective derived nucleic acid molecule flanked by one of (i) the first terminal region and (ii) the second terminal region of the forward strand or the reverse strand of the respective derived nucleic acid molecule (e.g., each sequence read includes only one adapter).

In some embodiments a terminal region of each respective sequence read in the plurality of sequence reads further comprises a molecular identifier (310) that is unique (e.g., a unique molecular identifier or UMI) to the derived nucleic acid molecule the respective sequence read was amplified from. For example, in some embodiments, the UMI identifies, for each sequence read (e.g., obtained from a sequencing reaction of an obtained target nucleic acid molecule and/or a PCR-amplified target nucleic acid molecule), the original target nucleic acid molecule from which the respective sequence read was derived. Thus, the UMI serves as an identifier for a respective target nucleic acid molecule where sequencing is performed for a plurality of target nucleic acid molecules (e.g., a plurality of nucleic acid fragments in a sequencing library). In some embodiments, a terminal region of each respective sequence read in the plurality of sequence reads further comprises a flow cell binding sequence (312). In some embodiments, as described above, the UMI (310) and/or the flow cell binding sequence (312) is ligated to the target nucleic acid molecule, prior to the obtaining the sequencing dataset, via a PCR reaction. In some embodiments, the UMI (310) and/or the flow cell binding sequence (312) is included in one or more single-stranded or double-stranded adapters.

In some embodiments, a terminal region of each respective sequence read in the plurality of sequence reads further does not comprise a unique molecular identifier (UMI).

For example, in some embodiments, the sequencing method is a targeted sequencing, and each respective sequence read in the plurality of sequence reads is flanked by at least a first terminal region comprising a UMI (e.g., the sequence read comprises at least one UMI). In some alternative embodiments, the sequencing method is a methylation sequencing, and each respective sequence read in the plurality of sequence reads i) is flanked by at least a first terminal region, and ii) does not comprise a UMI.

In some embodiments, the plurality of sequence reads comprises at least 1 sequence read, at least 2 sequence reads, at least 3 sequence reads, at least 4 sequence reads, at least 5 sequence reads, or at least 6 sequence reads.

In some embodiments, the sequencing provides non-duplex, paired end sequence reads (e.g., where only one strand of each derived nucleic acid molecule is captured by sequencing). See e.g., U.S. Pat. No. 7,601,499, entitled “Paired End Sequencing” and filed Jun. 6, 2006 and Campbell et al., 2009 Nat Genetics 40(6), 722-729 for descriptions of paired end sequencing methods. In such embodiments, there is a corresponding first population of sequence reads in the plurality of sequences reads that (i) were amplified from the respective forward strand of the corresponding derived nucleic acid molecule and (ii) includes the first terminal region ligated to the respective forward strand of the respective derived nucleic acid molecule. There is also a corresponding second population of sequence reads in the plurality of sequences reads that (i) were amplified from the respective forward strand of the corresponding derived nucleic acid molecule and (ii) includes the second terminal region ligated to the respective forward strand of the respective corresponding nucleic acid molecule. At least a first portion of each respective sequence read in the first population of sequence reads for the respective derived nucleic acid molecule is not complementary (e.g., non-overlapping) to any portion of any sequence read in the second population of sequence reads for the respective derived nucleic acid molecule. However, a second portion of each respective sequence read in the first population of sequence reads for the respective derived nucleic acid molecule is complementary (e.g., overlapping or stitched) to a portion of at least one sequence read in the second population of sequence reads for the respective derived nucleic acid molecule.

In some embodiments, the sequence provides duplex, paired end sequence reads (e.g., where both the forward and reverse strands of each obtained nucleic acid molecule is captured by two rounds of sequencing). See e.g., Schmitt et al., 2012 PNAS 109(36), 14508-15513 or Kennedy et al., 2014 Nat Protoc 9(11), 2586-2606 for a description of duplex sequencing. In such embodiments, there is a corresponding first population, second population, third population and fourth population of sequence reads in the plurality of sequence reads. In such embodiments, the first and second populations of sequence reads are amplified from the respective forward strand of the corresponding derived nucleic acid molecule. Conversely, the third and fourth populations of sequence reads are amplified from the respective reverse strand of the corresponding derived nucleic acid molecule. In such embodiments, the first and third populations of sequence reads include the first terminal region and the second and fourth populations of sequence reads include the second terminal region. In such embodiments, at least a first portion of each respective sequence read in the first and third population of sequence reads for the corresponding derived nucleic acid molecule is not complementary (e.g., non-overlapping) to any portion of any sequence read in the second or fourth population of sequence reads for the corresponding derived nucleic acid molecule. Similar to non-duplex, paired end sequencing, a second portion of each respective sequence read in the first and third population of sequence reads for the corresponding derived nucleic acid molecule is complementary (e.g., overlapping or stitched) to a portion of at least one sequence read in the second or fourth population of sequence reads for the corresponding derived nucleic acid molecule.

In some embodiments, the first and third populations of sequence reads in the above examples correspond to the “left reads” of the forward and reverse strands, respectively, of each target nucleic acid molecule, where a left read is produced by a first cycle of paired-end sequencing (e.g., an R1 sequencing reaction). The second and fourth populations of sequence reads correspond to the “right reads” of the forward and reverse strands, respectively, of each target nucleic acid molecule, where a right read is produced by a second cycle of paired-end sequencing (e.g., an R2 sequencing reaction).

In some embodiments, the first, second, third, and fourth populations of sequence reads can be distinguished based on a respective unique first, second, third, and fourth terminal region included in the non-overlapping portion of each sequence read that denotes at least the strand (e.g., forward or reverse) and/or the directionality (e.g., left or right orientation) of the sequence read.

In some embodiments, the plurality of sequence reads provides an average coverage of between 20× and 70,000× across the plurality of base reads 124. In some embodiments, for example when performing whole genome (bisulfite or non-bisulfite) sequencing, an average coverage rate of the plurality of sequence reads that are taken from a biological sample is at least 1×, 2×, 3×, 4×, 5×, 6×, 7×, 8×, 9×, 10×, at least 20×, at least 30×, at least 40×, at least 50×, at least 100×, or at least 200× across the genome (or across the first plurality of base reads) of the subject.

In some embodiments, for example when sequencing (methylation- or nonmethylation-based) using a targeted panel of genes is performed, an average coverage rate of the plurality of sequence reads that are taken from a biological sample of the subject is at least 200×, 200×, 500×, 1,000×, at least 2,000×, at least 3,000×, or at least 4,000×, at least 5,000×, at least 10,000×, at least 20,000×, at least 30,000×, or at least 50,000× across selected regions in the genome (or across the plurality of base reads) of the subject.

In some embodiments, the targeted panel of genes (e.g., and/or other selected regions in the genome of the subject) is within the range of 250 genes, within the range of 500 genes, within the range of 750 genes, within the range of 1000 genes, within the range of 2000 genes, within the range of 4000 genes, within the range of 6000 genes, or within the range of 8000 genes. In some such embodiments, the targeted assay looks for single nucleotide variants in the targeted panel of genes (e.g., and/or other selected regions in the genome of the subject), insertions in the targeted panel of genes, deletions in the targeted panel of genes, somatic copy number alterations (SCNAs) in the targeted panel of genes, or re-arrangements affecting the targeted panel of genes.

Selecting Features.

In some embodiments, the features for each base read of the plurality of base reads in the first sequencing dataset (e.g., for determining the consensus base call) are selected based on principal component analysis of training data. In some embodiments, a principal component analysis of training data is performed to identify 1 or more components, 2 or more components, 3 or more components, 4 or more components, 5 or more components, 6 or more components, 7 or more components, 8 or more components, 9 or more components, or 10 or more components. In some embodiments, the number of components is selected as the set of components that explain at least 20% of the variation, at least 30% of the variation, at least 40% of the variation, at least 50% of the variation, at least 60% of the variation, at least 70% of the variation, at least 80% of the variation, or at least 90% of the variation in the training data (e.g., where the variation is the variability observable in each original feature of the data).

For example, in some such embodiments, features are selected by identifying the variables that drive variation between components. In some such embodiments, features are selected from the loadings for each principal component that explain at least the threshold percentage of the variation in the training data.

In some embodiments, the features for determining the consensus base call are selected based on principal component analysis (PCA) of misclassified examples of training data. In some such embodiments, the selected features include one or more additional features for improving classification.

An example PCA method suitable for selecting one or more features for determining the consensus base call is sparse PCA (SPCA). See Zou et al., 2006 J Computing and Graph. Stats. 15(2), 265-286. Typical PCAs compress all of the original features from a dataset into one or more linear combinations (e.g., components). This makes it difficult to interpret which original features should be selected. SPCA seeks to resolve this issue by using sparse principal component loadings to determine principal components that are composed of a few features (e.g., up to 1 feature, up to 2 features, up to 3 features, up to 4 features, up to 5 features, or up to 6 features). This makes it easier to select features for downstream analysis. This is only for purposes of illustration, and is not intended to limit the methods used in feature selection. An overview of other possible feature selection methods is provided by Saeys et al., 2007 Bioinformatics 23(19), 2507-2517.

In some embodiments, the features used for determining the consensus base call are selected using an unsupervised dimension reduction method. In some embodiments, the features used for determining the consensus base call are selected using a supervised dimension reduction method. For example, in some embodiments, feature selection is performed by using a support vector machine, random forest, sequential backward selection, kernel PCA, linear regression, k nearest neighbor, or combination thereof. This list of feature selection methods is intended to be illustrative and is not limiting. Other feature selection methods known in the art could also be suitable for use with the methods described herein. For example, feature selection methods suitable for use with the methods described herein are discussed in Batenhagen et al., BMC 2010 Bioinformatics 11, 567; Sorzano et al., 2014 arXiv:1403.2877; and Diaz et al., 2016 Bioinformatics 32(14), 2219-2220.

In some embodiments, misclassification is more likely for base reads with low bag depth at a first bag depth position (e.g., reads with a sequencing depth less than a minimum threshold of base reads), for base reads in non-duplexed positions, for base reads at terminal positions within sequence reads, or for base reads that are part of specific trinucleotide contexts (e.g., each and every possible three nucleotide permutation of A, T, C, and G or each and every possible three nucleotide permutation of A, U, C, and G). In some such embodiments, the aforementioned minimum threshold of base reads is less than 1, less than 2, less than 3, less than 4, or less than 5 base reads.

Additional Features.

In some embodiments, the sequencing dataset further comprises one or more additional features for each base read at the first base position.

In some embodiments, the one or more additional features comprise a bag depth count of a total number of base reads at the first base position. A bag depth refers to the sequencing depth of a base position (e.g., a number of sequence reads that provide information (e.g., base reads) for an individual base position). In some embodiments, the bag depth count comprises at least 1 base read, at least 2 base reads, at least 3 base reads, at least 4 base read, at least 5 base reads, at least 6 base read, at least 7 base reads, at least 8 base reads, at least 9 base reads, or at least 10 base reads for the first base position.

In some embodiments, the one or more additional features comprise a low bag depth indication for when the total number of base reads is below a minimum threshold. In some embodiments, the minimum threshold is less than 2, less than 3, less than 4, or less than 5 base reads.

In some embodiments, the one or more additional features comprise a distance of the base read relative to an end of its respective sequence read. For example, in some embodiments, the base position of the base read is compared to the total length of the respective sequence read and the resulting difference comprises the distance of the base read to an end of the sequence read. In some embodiments, the distance of the base read is a symmetric distance. For example, the distance comprises a whole number referring to the distance from the closest end of the sequence read to the base position. For example, for each of the following sequence reads, the distance to the closest end of the respective base position (e.g., the ‘A’) is indicated.

For the sequence read, 5′-XXXXXAXXX-3′, the distance is 3 (e.g., the base position of the ‘A’ is 6 and the length of the sequence read is 9, and six is subtracted from nine, leaving three). For the sequence read, 5′-XXXXAXXXX-3′, the distance is 4 (e.g., where this is an example of symmetric distance, where the ‘A’ is equidistant from the two ends). For the sequence read, 5′-XXXXXXXAX-3′, the distance is 1.

In some embodiments, the distance of the base read is an asymmetric distance. For example, the distance comprises an indication of a distance of a base read relative to the start (e.g., the 5′ end) of its respective sequence read. In some embodiments, the one or more additional features comprise an indication of a distance of a base read relative to the stop (e.g., the 3′ end) of its respective sequence read. In some such embodiments, the distinction between bases located closer to the start and the stop ends of their respective sequence read can be used to assign varying weights and/or penalties based on the quality of the sequencing reaction at different ends of the sequence read (e.g., where sequencing quality is higher at the start end and lower at the stop end).

In some embodiments, the one or more additional features comprise a terminal base indication indicating whether the base read is for a base position that is an edge or a non-edge base (e.g., is an end or a non-end base position) of a respective sequence read. For example, in some embodiments, the terminal base indication is a binary indication of whether the base position is an end or non-end position. For example, in the sequence read 5′-XXXXXAXXX-3′, the terminal base indication is 0, indicating that the base position of the ‘A’ is a non-end base position. Conversely, for the sequence reads 5′-AXXXXXXXX-3′ or 5′-XXXXXXXXA-3′, the terminal base indication is 1, indicating that the base position of the ‘A’ is an end base position.

In some embodiments, the terminal base indication indicates whether the base read is for a base position that is within a threshold distance from a read-edge. In some such embodiments, the threshold distance is 1, 2, 3, 4, 5, 6, 7, 8, 9, 10, 11, 12, 13, 14, 15, 16, 17, 18, 19, 20, or more than 20 base pairs from a read-edge. For example, a terminal base indication can be a binary indication of whether the base position is within 3 base pairs from an end position, such that, in the sequence read 5′-XAXXXXXXX-3′, the terminal base indication is 1.

In some embodiments, the one or more additional features comprise a duplex status indicating whether the base read is associated with a duplex or non-duplex read of the first base position. In such embodiments, the duplex status is a binary indication (e.g., a “0” indicates that the base read is associated with a non-duplex sequence read, and a “1” indicates that the base read is associated with a duplex sequence read). The method of duplex dna sequencing is reviewed by Kennedy et al., 2014 Nat Protoc 9(11), 2586-2606, and involves tagging each duplex DNA fragment with a respective unique molecular identifier that is random and double-stranded.

In some embodiments, the one or more additional features comprise a certain trinucleotide context indicating when the base read is one of three predetermined adjacent nucleotide bases (e.g., the certain trinucleotide context indicates at least one adjacent nucleotide base upstream of the base position and at least one adjacent nucleotide base downstream of the base position). In such embodiments, one or more respective sequence reads in the plurality of sequence reads in the sequencing dataset further includes a base call for an upstream base position from the respective base position and a base call for a downstream position from the respective base position. For example, a respective sequence read may comprise the base reads AAA, AAT, AAC, AAG, TAA, TAT, TAC, TAG, CAA, CAT, CAC, CAG, GAA, GAT, GAC, or GAG (e.g., where the base position in the middle, the ‘A,’ is the base position for which a consensus base position is being determined. In some embodiments, a respective sequence read comprises any permutation of three nucleic acid bases selected from the set of ‘A,’ ‘T,’ ‘C,’ and ‘G.’ In some embodiments, a respective sequence read comprises any permutation of three nucleic acid bases selected from the set of ‘A,’ ‘U,’ ‘C,’ and ‘G.’

In some embodiments, the one or more additional features comprise an overlap status indicating whether the base read is a stitched or non-stitched read. For example, in some embodiments, such as when two or more sequence reads from paired-end sequencing map to the same genomic region, then some part of the two or more sequence reads is overlapping and the two or more sequence reads can be merged along the overlapping sequence (e.g., following protocols outlined in, for example, Al-Ghalith et al., 2018, mSystems 3(3), e00202-17 and Zhang et al., 2014, Bioinformatics 30(5), 614-620). In such embodiments, an overlap status comprising a binary indication of whether the position of the base read is from a stitched (e.g., overlapping) or non-stitched (e.g., non-overlapping) part of the two or more sequence reads is provided (e.i., the binary indication is 1 for when the base position is from a stitched region or 0 for a non-stitched region). The overlap status provides more information on the quality of the base read, since each base read from a base position from an overlapping region has more support (e.g., increased read depth) than a base read from a non-overlapping region.

In some embodiments, the one or more additional features comprise an ambiguous strand base count at the base read. In some embodiments, ambiguous strand base counts refer to cases where the orientation (e.g., forward or reverse status) of the sequence read is not known or is not welldefined. This could occur for a small proportion of sequence reads that map to regions with inverted tandem duplications, or for regions with palindromic sequences; see Reams et al., 2015, Cold Spring Harb Perspect Biol 7(2), a016592). This additional feature will only be relevant for a small set of sequence reads in the plurality of sequence reads. In some embodiments, the ambiguous strand base count represents a count of sequence reads

In some embodiments, the one or more additional features comprise a mapping quality (e.g., an estimated accuracy of the mapping of a respective sequence read to the base position) of the base read. For example, a mapping quality score can reflect the alignment accuracy of a sequence read to a particular base position (e.g., a mapping quality score could range from 0-100. See e.g., Li et al., 2008, Genome Res. 18(11), 1851-1858. The quality of the overall mapping of sequence reads in turn affects the confidence of determining a consensus base call.

In some embodiments, the one or more additional features comprise a distance of the base read from a homopolymer, insertion, or deletion. For example, in some embodiments, the location of the base position of the base read in a reference genome is compared to a polymorphism location (e.g., a base position of one or more homopolymers, insertions, or deletions) in the reference genome to determine a distance between the base position and the polymorphism location. The distance refers to a number of base positions between the base read and the polymorphism (e.g., the distance comprises at least 1 base position, at least 2 base positions, at least 3 base positions, at least 4 base positions, at least 5 base positions, at least 10 base positions, at least 15 base positions at least 20 base positions, at least 30 base positions, at least 40 base positions, at least 50 base positions, at least 60 base positions, at least 70 base positions, at least 80 base positions, at least 90 base positions, at least 100 base positions, at least 100 base positions, at least 200 base positions, at least 300 base positions, at least 400 base positions, or at least 500 base positions).

In some embodiments, the one or more additional features comprise one or more interaction features between or within duplex strands. Interaction features are those that are determined based on at least two other features. For example, base quality scores are separately recorded for positive and negative strands. These positive and negative strand quality scores for a particular base location can be combined (e.g., multiplied) into an interaction feature between or within duplex strands. For example, consider one implementation having a count ‘n’ of positive strand sequence reads with a quality score of 41 for base A at position L, and a count ‘m’ of negative strand sequence reads with a quality score of 41 for base A at position L. A corresponding “between-duplex-strands” interaction feature would be the result of n multiplied by m. In some embodiments, one or both of n and m are zero, leading the corresponding between-duplex-strands interaction feature to be zero. As an example of determining an interaction feature within duplex strands, consider the above example implementation further having a count ‘p’ of positive strand sequence reads with a quality score of 41 for base G at position L. This value p is then multiplied with n (e.g., count “n” of positive strand sequence reads with a quality score of 41 for base A at position L), thus providing the “within-duplex-strands” interaction feature. These interaction features can reveal sequencing errors. If there were, for instance, a PCR error within the bag, and both A and G base calls were made for a particular position (e.g., L), this interaction feature would have a nonzero value.

In some embodiments, the one or more additional features comprise a sequencing read identifier (e.g., a read identifier) indicating the direction of sequencing (e.g., “read-side”) of the respective sequence read of the base read. In some embodiments, this additional feature is available for sequence reads that are derived from either duplex or non-duplex paired-end sequencing. I n some embodiments, the sequencing read identifier is binary (e.g., a 1 indicates that the respective sequence read is from a left read (e.g., an R1 read) of the target nucleic acid molecule and a 0 indicates that the respective sequence read is from a right read (e.g., an R2 read) of the target nucleic acid). For example, the sequencing read identifier can be used to distinguish between and/or assign weights or penalties to sequencing errors specific to read-side (e.g., where higher error rates are observed at the 3′ ends of right reads compared to the 3′ ends of left reads).

In some embodiments, the one or more additional features comprise a sequencing cycle identifier indicating the sequencing cycle during which the respective base read was obtained (e.g., between 1 and 151). For example, a sequencing cycle identifier can be used to distinguish between and/or assign weights or penalties to sequencing errors specific to sequencing cycle (e.g., where higher error rates are observed in the first cycle compared to all subsequent cycles).

In some embodiments, the one or more additional features comprise a bag number identifying one or more bags corresponding to a respective target nucleic acid molecule; a chromosome number identifying the chromosome of a reference genome to which the target nucleic acid molecule is aligned; a reference base position number identifying the base position of a reference genome to which the respective base read is aligned; and/or a reference base identity indicating the base identity, at the respective base position, of a reference genome to which the respective base read is aligned.

In some embodiments, the one or more additional features comprise a reverse-complement identifier, indicating where features contain overlapping or repetitive information (e.g., where the same feature is represented in both a left read and a right read).

In some embodiments, the one or more additional features comprises an A-G or T-C base indicator (e.g., where higher error rates are observed due to A-G and/or T-C base damage following bisulfite treatment).

In some embodiments, the number of features selected for the first sequencing dataset is predetermined. In some alternative embodiments, the number of features selected for the first sequencing dataset is determined by a user or practitioner based on one or more optimization assays (e.g., such that the data best fits the model without overfitting). In some embodiments, the features selected for the first sequencing dataset comprises any of the above features, additional features, and/or any combinations or substitutions thereof as will be apparent to one skilled in the art.

Combinations of Features

In some embodiments, the sequencing dataset includes a respective plurality of features for each base read of the plurality of base reads. In some embodiments, the respective plurality of features for a first base read in the plurality of base reads differ from the respective plurality of features for a second base read in the plurality of base reads (e.g., one or more of the base reads in the plurality of base reads in has a different plurality of features). Different possible combinations of features are described below.

In some embodiments, the sequencing dataset comprises a respective plurality of features (e.g., two or more of the features set forth in Table 1). In some embodiments, the plurality of features includes some or all of the features listed in Table 1. For example, in some embodiments, the plurality of features includes 2, 3, 4, or all 5 of the features listed in Table 1. In one embodiment, the plurality of features includes at least features 1-5 as provided in Table 1.

TABLE 1
Exemplary Features for a respective base read

| | Exemplary Features |
|---|---|
| 1 | a nucleotide base of the respective base read |
| 2 | a read quality score associated with the respective base read |
| 3 | a strand identifier indicating whether the respective sequence |
| | read of the respective base read corresponds to a forward or a |
| | reverse strand of the target nucleic acid molecule |
| 4 | a trinucleotide context based on three consecutive nucleotide |
| | bases including the respective base read |
| 5 | a confidence score associated with the trinucleotide context |

It is contemplated that, in some embodiments, any one or more of the features provided in Table 1 will not be included in the plurality of features. For example, in some embodiments, a particular feature will be informative with regard to a specific target nucleic acid molecule but not for another target nucleic acid molecule.

Accordingly, it is contemplated that the plurality of features include at least any two of the features provided in Table 1. For brevity, all possible combinations of features are not specifically detailed here. However, the skilled artisan will easily be able to envision any particular subset of the plurality of features provided in Table 1. For example, in some embodiments, the respective plurality of features for a respective base read of the plurality of base reads comprises at least a nucleotide base of the respective base read and a read quality score associate with the respective base read. Likewise, the skilled artisan may know of other features (e.g., that are not included in Table 1) that may be combined with any subset of the plurality of features in the sequencing dataset and used according to the methods described herein.

In some embodiments, the sequencing dataset further comprises one or more of the additional features listed in Table 2 (e.g., the respective plurality of features for each respective base read further comprises one or more of the additional features). In some embodiments, the plurality of features further includes some or all of the additional features listed in Table 2. For example, in some embodiments, the plurality of features further includes 2, 3, 4, 5, 6, 7, 8, 9, 10, 11, or all 12 of the features listed in Table 2.

TABLE 2
Exemplary Additional Features for a Respective Base Read

| | Exemplary Additional Feature |
|---|---|
| 1 | a bag depth count of a total number of base reads at the first |
| | base position |
| 2 | a first bag depth indicating when the total number of base reads |
| | is below a minimum threshold |
| 3 | a distance of a base read relative to an end of a respective |
| | sequence read in the plurality of sequence reads |
| 4 | a terminal base indicating whether the base read is an edge or |
| | a non-edge base |
| 5 | a duplex status indicating whether the base read is associated |
| | with a duplex or non-duplex read of the first base position |
| 6 | a certain trinucleotide context indicating when the base read |
| | is one of three predetermined adjacent nucleotide bases |
| 7 | an overlap status indicating whether the base read is a |
| | stitched or non-stitched read |
| 8 | an ambiguous strand base count at the base read |
| 9 | a mapping quality of the base read |
| 10 | a distance of the base read from a homopolymer, insertion, |
| | or deletion |
| 11 | one or more interaction features between or within duplex strands |
| 12 | a read direction indicating whether the respective sequence read |
| | of the base read is a forward or reverse read of the target nucleic |
| | acid molecule |

It is contemplated that, in some embodiments, any one or more of the additional features provided in Table 2 will not be included in the sequencing dataset. For example, in some embodiments, a feature will be informative with regard to a specific target nucleic acid molecule but not for another target nucleic acid molecule. Accordingly, it is contemplated that the additional features included in the sequencing dataset may be any sub-set of features provided in Table 2. Likewise, the skilled artisan may know of other features not provided in Table 2 that may be combined with any subset of the features provided in Table 2 and/or the features selected from Table 1 to form the sequencing dataset used in the methods described herein. In some embodiments, any combination of (i) any two features selected from Table 1 plus (ii) any one or more features selected from Table 2 is used to form the sequencing dataset.

Bagging Reads.

Referring to block 206, in some embodiments, the first plurality of sequence reads define a pileup of sequence reads (e.g., in a bag of sequence reads) having the same unique molecular identifier (e.g., UMI) or the same genomic position (see, Definitions: “Bag”). In some embodiments, the first plurality of sequence reads has both the same UMI and the same genomic position. This is particularly important for embodiments where there are a few (e.g., less than 5, less than 10, less than 20, less than 50, less than 100, or less than 200) UMIs and many (e.g., billions) of sequence reads (e.g., where one or more sequence reads having the same UMI in common is not enough to ensure that the one or more sequence reads map to the same genomic sequence or region). In some embodiments, one or more sequence reads in the plurality of sequence reads lacks a UMI. In such embodiments, such sequence reads can be bagged based on common start and end positions (e.g., after alignment of the one or more sequence reads).

For example, as illustrated in FIG. 4, one or more sequence reads with the same UMI (e.g., as indicated by a UMI sequence in one or both of 320 and 322) can define a first pileup of sequence reads. In some embodiments, the first pileup of sequence reads is also aligned (e.g., to a reference genome of the subject). In FIG. 4, base reads for one base position are considered. Of the five sequence reads with the same UMI, four have a base read of ‘G’ at the respective base position (e.g., 402-1, 402-2, 402-3, and 402-5), and the remaining sequence read has a base read of ‘A’ (402-4) for the respective base position. In some embodiments, each respective base read has an associated base read quality score (e.g., phred quality scores for the sequence reads) in addition to a nucleotide base.

In some embodiments, the first plurality of sequence reads of the target nucleic acid molecule is bagged using duplicate marking. As used herein, “duplicate marking” refers to the process of identifying sequence reads that are derived, via a sequencing method, from the same original nucleic acid molecule or nucleic acid fragment.

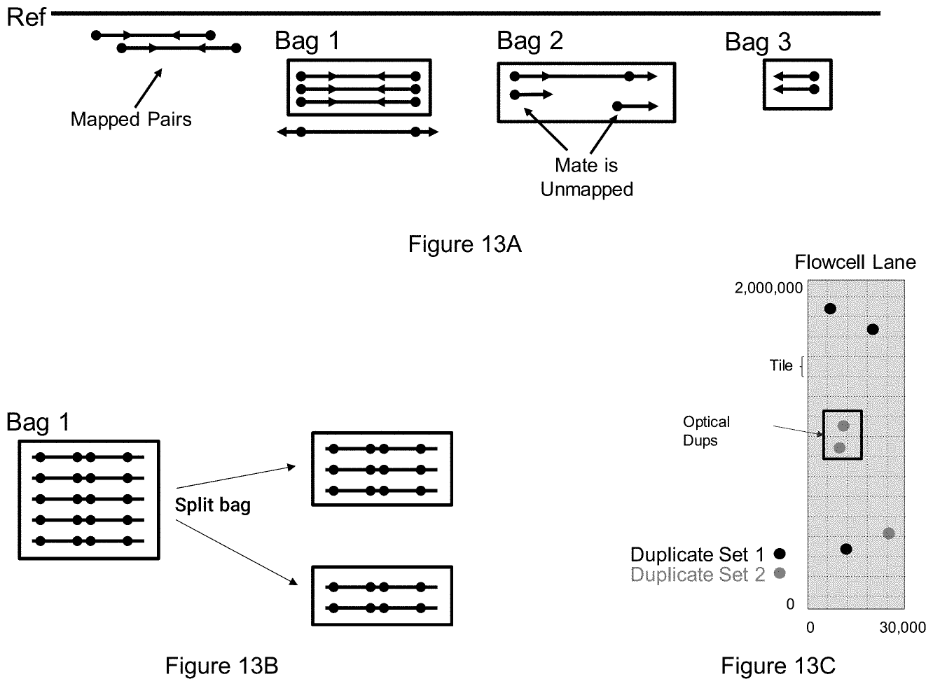

In some such embodiments, two sequence reads are marked as duplicates if they have i) equal 5′ start positions when aligned to a reference genome and ii) the same strand polarity (e.g., forward or reverse). For paired-end sequencing data, two read-pairs are marked as duplicates when i) the left reads of each read pair are duplicates and ii) the right reads of each read pair are duplicates. FIG. 13A illustrates examples of bagging sequence reads. Bag 1, Bag 2 and Bag 3 illustrate duplicate marking for subsets of sequence reads that are aligned to the same region of a reference genome (“Ref”). Mapped read-pairs are indicated by double arrowheads within sequence reads, whereas unmapped single reads are indicated by single arrowheads. In some embodiments, as shown in Bag 2 of FIG. 13A, one or more sequence reads are bagged if they have i) equal 3′ stop positions when aligned to a reference genome and ii) the same strand polarity (e.g., forward or reverse).

In some embodiments, as illustrated in FIG. 13B, a first bag comprising a first plurality of sequence reads is subdivided into two or more split bags, each split bag comprising a subset of the sequence reads in the first bag of sequence reads. Splitting bags can be performed to preserve unique sequence reads, including sequence reads that have undergone sequencing errors and/or unique sequence reads that are bagged together by chance. For example, in some embodiments, between 1-2% of unique sequence reads are marked as duplicates based on genomic position at a sequencing depth of 1000×. Erroneously marked sequence reads preserved in separate bags can be used for error correction and/or improved alignment and coverage in downstream analysis. For example, in some implementations, splitting a parent bag based on a methylation state distance threshold of 8 generates a first child bag comprising 99.1% of read pairs and a second child bag comprising 0.9% of read pairs from the parent bag, and improves alignment of all read pairs from the parent bag by increasing unique coverage by 1%.

In some embodiments, bag splitting is performed based on a methylation distance metric determined using a comparison of methylation states between two or more sequence reads in a pileup of sequence reads. In some embodiments, bag splitting is performed for two or more sequence reads having a methylation distance metric between 1 and 20, between 3 and 15, or between 6 and 10. In some embodiments, bag splitting is performed when the methylation distance metric is 1, 2, 3, 4, 5, 6, 7, 8, 9, 10, 11, 12, 13, 14, 15, 16, 17, 18, 19, 20, or more than 20.

In some embodiments, determining the methylation distance metric (e.g., CpG distance metric) comprises tallying the number of methylation state discrepancies between an alternate sequence read and one or more “ground truth” sequence reads. For example, where a first R1 sequence read (R1-A) comprises a methylated state at a respective CpG site and a second R1 sequence read (R1-B) comprises an unmethylated state at the same respective CpG site, the number of methylation state discrepancies between the ground truth sequence read and the alternate sequence read is 1, and the CpG distance metric is 1.

In some embodiments, determining the CpG distance metric comprises using a matched sequence read in a read-pair (e.g., obtained from paired-end sequencing) to provide stitched support for a methylation state discrepancy between the alternate sequence read and the one or more “ground truth” sequence reads. For example, where a first R1 sequence read (R1-A) comprises a methylated state at a respective CpG site, a second R1 sequence read (R1-B) comprises an unmethylated state at the same respective CpG site, and a R2 sequence read that is the paired-end sequencing mate of the second R1 sequence read (R2-B) comprises an unmethylated state at the same respective CpG site, then the number of methylation state discrepancies between the ground truth sequence read and the alternate sequence reads is 2, and the CpG distance metric is 2.

In some embodiments, the one or more “ground truth” sequence reads are two sequence reads in a first read-pair (e.g., obtained from paired-end sequencing), and determining the CpG distance metric comprises using a matched sequence read in a second read-pair to provide stitched support for a methylation state discrepancy between the alternate sequence read and each of the two ground truth sequence reads. For example, where a first R1 sequence read (R1-A) and its paired-end sequencing mate (R2-A) both comprise a methylated state at a respective CpG site, and where a second R1 sequence read (R1-B) and its paired-end sequencing mate (R2-B) both comprise an unmethylated state at the same respective CpG site, then the number of methylation state discrepancies between each of the ground truth sequence reads R1-A and R2-A and each of the alternate sequence reads R1-B and R2-B is 4, and the CpG distance metric is 4.

In some embodiments, determining the CpG distance metric between two sets of read-pairs comprises calculating a sum of the CpG distance metrics for each CpG site in a plurality of CpG sites. For example, where a first R1 sequence read (R1-A) and its paired-end sequencing mate (R2-A) both comprise a methylated state at a first CpG site and a methylated state at a second CpG site, and where a second R1 sequence read (R1-B) and its paired-end sequencing mate (R2-B) both comprise an unmethylated state at the first CpG site and an unmethylated state at the second CpG site, then the number of methylation state discrepancies between each of the ground truth sequence reads R1-A and R2-A and each of the alternate sequence reads R1-B and R2-B is 8 (4+4), and the CpG distance metric is 8.

In some embodiments, a CpG site is assigned a methylation state discrepancy value of 1 where the discrepancy comprises a methylated (“M”) status and an unmethylated (“U”) status. In some embodiments, a CpG site is assigned a methylation state discrepancy value of 0 where the discrepancy comprises at least one variant (“V”) status or at least one ambiguous (“A”) status.

In some embodiments, the first plurality of sequence reads of the target nucleic acid molecule is bagged based on an identification of optical duplicates. As used herein, “optical duplicates” refer to two or more sequence reads having coordinate positions that can be visualized in a single flow cell lane. In some such embodiments, a first sequence read and a second sequence read are determined to be optical duplicates where the first sequence read and the second sequence read i) are both contained within a single bag; ii) are both contained in matching read groups and libraries; iii) comprise matching R1 and R2 orientations; and iv) satisfy a threshold distance with respect to the x-axis and y-axis position of the first and the second sequence read when visualized in a flow cell lane.

In some embodiments, the threshold distance along the x-axis is 30,000 or less, 25,000 or less, 20,000 or less, 15,000 or less, 10,000 or less, 5,000 or less, 1,000 or less, 500 or less, or 100 or less. In some embodiments, the threshold distance along the y-axis is 2 million or less, 1.5 million or less, 1 million or less, 500,000 or less, 250,000 or less, 100,000 or less, 50,000 or less, or 10,000 or less.

Other methods of identifying and marking duplicates are possible. For further details on methods of identifying and marking duplicates and methods of bagging sequence reads, see, for example, U.S. application Ser. No. 16/839,469, “Methylation-based False Positive Duplicate Marking Reduction,” filed Apr. 3, 2020, which is hereby incorporated herein by reference in its entirety.

Filtering and Aligning Reads.

In some embodiments, the method further comprises removing from the plurality of sequence reads of the target nucleic acid molecule each base position in the plurality of base positions that satisfies a filtering criterion.

In some embodiments, the filtering criterion for each base position is a threshold number of alternative nucleotide base identities for the respective base position (e.g., variant positions). In some embodiments, the threshold number of alternative nucleotide base identities is 2 or 3. For example, in some such embodiments, each base position in the plurality of base positions comprising two or more alternative base read identities in the first plurality of sequence reads is removed from the plurality of base positions of the target nucleic acid molecule. In some embodiments, the filtering removes single nucleotide variant positions from the plurality of base positions of the target nucleic acid molecule.

In some embodiments, the filtering criterion for each base position is an “unknown” true base identity for the base position in a reference genome. For example, in some such embodiments, each base position in the plurality of base positions corresponding to a base position in a reference genome that lacks a base identity (e.g., a “true” base identity) is removed from the plurality of base positions of the target nucleic acid molecule. In some such embodiments, the true base identity is determined using a reference genome constructed from a training dataset. In some embodiments, the true base identity is determined using a reference genome constructed from a test dataset.

In some embodiments, the filtering criterion for each base position is a “homozygous variant” identity for the base position compared to a reference genome. As used herein, the term “homozygous variant” refers to a base position where each base read in the plurality of base reads comprises the same base identity, where the base identity of each base read differs from the base identity at the respective base position in the reference genome. For example, a homozygous variant is identified where, for a respective base position, each sequence read in a plurality of sequence reads comprises a base identity of “A” and a reference genome comprises a base identity of “G.” In some such embodiments, the reference genome is determined based on a sequencing (e.g., a targeted sequencing and/or a targeted methylation sequencing) of a first chromosome of a subject, and the homozygous variant is determined based on a sequencing (e.g., a targeted sequencing and/or a targeted methylation sequencing) of a second chromosome of the subject.

In some embodiments, the filtering criterion for each base position is, for each respective sequence read in the plurality of sequence reads, a positive strand nucleotide base identity of cytosine and a negative strand nucleotide base identity of guanine (e.g., a methylation site). For example, in some embodiments, removal of such methylation sites reduces potential error in base calling by removing base positions affected by bisulfite treatment (e.g., where cytosine bases are converted to thymine bases). In some alternative embodiments, potential methylation sites are not removed from the plurality of base positions of the target nucleic acid molecule, and the method further comprises using an estimated error rate to correct for miscalled cytosines resulting from cytosine to thymine bisulfite conversion. In some such embodiments, the estimated error rate is determined based on the raw (e.g., uncalibrated) base calling error rate for adenine, guanine, and thymine. In some such embodiments, the estimated error rate is an average of the base calling error rate for adenine, guanine, and thymine. In some alternative embodiments, the estimated error rate is determined by linearly interpolating the error rate of miscalled cytosines into one or more reference error rates corresponding to A, G, and/or T.

In some embodiments, the filtering criterion for each base position is a coordinate mismatch for the base position in the plurality of base positions, mapped to a reference genome. In some embodiments, the coordinate mismatch is determined using by mapping each sequence read in the first plurality of sequence reads to a reference genome and identifying each base position captured in the first plurality of sequence reads that is not captured in the reference genome, where the first plurality of sequence reads is obtained using a first sequencing method and the reference genome is obtained using a second sequence method that is different from the first sequencing method. For example, in some such embodiments, the filtering removes, from a plurality of base positions obtained from a methylation sequencing, each base position that is not included in a reference genome obtained from a targeted sequencing (e.g., each inserted base position).

In some embodiments, the filtering criterion for each base position is selected based on a consensus alignment for the plurality of sequence reads comprising the respective base position.

In some embodiments, the method further comprises obtaining a mapping string (e.g., CIGAR string) for each sequence read in a plurality of sequence reads of the target nucleic acid molecule, thus obtaining a plurality of mapping strings. In some embodiments, the obtaining a mapping string is performed prior to obtaining the sequencing dataset. Each mapping string in the plurality of mapping strings (i) is determined by an alignment of the respective sequence read to a reference genome and (ii) comprises a plurality of encodings, where each encoding in the plurality of encodings represents a coordinate match status of one or more base positions in the respective sequence read compared with the reference genome. In some embodiments, each encoding in the plurality of encodings is selected from the group consisting of match “M,” insertion “I,” deletion “D,” skipped “N,” soft-clipping “S,” and hard-clipping “H”. In some such embodiments, each encoding in the plurality of encodings further comprises an indication of a number of base positions in the one or more base positions that have the respective coordinate match status. In some embodiments, each base position in the plurality of base positions of the target nucleic acid molecule that fails to satisfy a selection criterion is removed from one or more sequence reads in the plurality of sequence reads, where the selection criterion is a measure of central tendency of the number of observations of one or more encodings for the respective base position, across each sequence read in the plurality of sequence reads that align to a region of the reference genome spanning at least the respective base position. In some embodiments, the measure of central tendency is a median or a mode.

In the above embodiments, the alignment of the respective sequence read to the reference genome is determined using the base identity of each base position in the respective sequence read, and each mapping string is a representation of the alignment of each sequence read to the reference genome, where the representation is based on the coordinates, but not the base identity, of each base position in the respective sequence read. The plurality of encodings for each mapping string represents the number of consecutive bases that match or do not match the coordinate sequence of the reference genome. See, Dunning, “Understanding Alignments,” 2017, which is hereby incorporated herein by reference in its entirety, for further details regarding CIGAR strings.

An example of mapping string encodings is provided in the sequence “M99I5,” where a first encoding comprises the coordinate match status “M” (match) and further comprises an indication of the number of base positions that have the match status “M” (e.g., 99). A second encoding comprises the coordinate match status “I” (insertion) and further comprises an indication of the number of base positions that have the match status “I” (e.g., 5). Thus, the mapping string M99I5 denotes an alignment where the first 99 base pairs of the sequence read are present in the reference genome, and the last 5 base pairs of the sequence read are inserted as compared to the reference genome. In some embodiments, an encoding comprises the value “0” where no base positions have the respective coordinate match status (e.g., no insertions =I0).

The mapping string encodings “S” (soft-clipping) and “H” (hard-clipping), as used herein, refer to methods of processing sequence reads for alignment to a reference genome. In soft-clipping, portions of the sequence read (e.g., at the terminal ends of the sequence read) that map poorly to the reference genome based on base identity are excluded from the alignment (e.g., ignored). In hard-clipping, portions of the portions of the sequence read (e.g., at the terminal ends of the sequence read) that map poorly to the reference genome based on base identity are excluded from the alignment (e.g., ignored), and those portions are further removed from the alignment record (e.g., truncated such that those portions of the sequence reads are fully removed from the alignment file, such as a BAM or SAM file). In some embodiments, a penalty for each soft-clipped and/or hard-clipped base is applied to any confidence score or alignment score generated from the alignment, thus accounting for potential misalignment resulting from such softclipping and/or hardclipping. In some embodiments, softclipping is performed for each sequence read to trim one or more terminal regions (e.g., adapters) from the respective sequence read.

In some embodiments, each base position in the plurality of base positions of the target nucleic acid molecule that fails to satisfy a selection criterion is removed from one or more sequence reads in the plurality of sequence reads, where the selection criterion is, for the respective base position, a threshold percentage of base reads comprising the “M” (match) coordinate match status. For example, in some such embodiments, the threshold percentage of bases is 100%, such that each base position that does not exhibit 100% concordance between the plurality of sequence reads and the reference genome is removed from the plurality of base positions.

Alternatively, in some embodiments, the selection criterion is a measure of central tendency of the number of observations of one or more encodings for the respective base position, across each sequence read in the plurality of sequence reads that align to a region of the reference genome spanning at least the respective base position. In some embodiments, the measure of central tendency is a median or a mode (e.g., a majority vote).

For example, in some embodiments, a mode of observations of one or more encodings for a respective base position is determined by, for each respective sequence read in a plurality of sequence reads comprising the respective base position, creating a corresponding mapping string (e.g., a CIGAR string) determined by an alignment of the respective sequence read to a reference genome. The numbers of each unique encoding at the respective base position are tallied, and the encoding that is represented the highest number of times at the respective base position is selected, thus designating a consensus encoding for the plurality of sequence reads. In an example implementation, four individual sequence reads may comprise the following encodings at a certain base position: I1, I2, I2, and I3, where each encoding denotes an insertion of 1, 2, 2, and 3 base pairs, respectively, in each sequence read, compared to a reference genome. The numbers of each unique encoding at the respective base position are I1:1; I2:2; and I3:1; and the encoding that is represented the highest number of times is I2. The mode encoding thus designates, for the consensus alignment, an insertion of 2 base pairs at the respective base position.

In some alternative embodiments, a median of observations of one or more encodings for a respective base position is determined by, for each respective sequence read in a plurality of sequence reads comprising the respective base position, creating a corresponding mapping string (e.g., a CIGAR string) determined by an alignment of the respective sequence read to a reference genome. The values of each unique encoding at the respective base position are ranked, and the encoding that is represented at the median of the ranked encodings is selected, thus designating a consensus encoding for the plurality of sequence reads. In an example implementation, five individual sequence reads may comprise the following encodings at a certain base position: I1, I1, I2, I3 and I3, where each encoding denotes an insertion of 1, 1, 2, 3, and 3 base pairs, respectively, in each sequence read, compared to a reference genome. The ranked values of each unique encoding at the respective base position are 1, 1, 2, 3, and 3; and the encoding that is represented at the median of the ranked encodings is I2. The median encoding thus designates, for the consensus alignment, an insertion of 2 base pairs at the respective base position.

In some embodiments, each sequence read in the plurality of sequence reads is modified to match the coordinate mapping denoted by the consensus alignment (e.g., as designated by the median and/or mode encoding). In some embodiments, the modification comprises adding one or more base positions. In some alternative embodiments, the modification comprises deleting one or more base positions. For example, as described above, where the consensus alignment denotes an insertion of 2 base pairs, a first sequence read comprising an encoding of “I1” would be modified to incorporate an extra base at the respective position. In some such embodiments, the inserted base is represented by a dummy base. In some embodiments, where the consensus alignment denotes an insertion of 2 base pairs, a second sequence read comprising an encoding of “I3” would be modified to include only the first two bases out of the original span of three bases, such as by truncating the insertion to exclude the third base.

In some embodiments, deletion of one or more base positions by the abovementioned methods and embodiments is used to process sequence reads (e.g., by soft-clipping and/or hard-clipping). For example, in some embodiments, the consensus alignment is used to determine the number of bases to be excluded from the alignment (e.g., the leading soft-clip length) and thus determine the start position of the alignment.

In some embodiments, consensus alignments are performed over a plurality of sequence reads comprising both R1 and R2 sequence reads, where the R1 and R2 sequence reads comprise matching pre-clipped start positions, as determined based on an alignment to a reference genome.

In some embodiments, the removing from the plurality of sequence reads of the target nucleic acid molecule each base position in the plurality of base positions that satisfies a filtering criterion, where the filtering criterion for each base position is selected based on a consensus alignment for the plurality of sequence reads comprising the respective base position, is performed in order to improve the sensitivity and/or accuracy of downstream probe detection analysis.

In some embodiments, the removing from the plurality of sequence reads of the target nucleic acid molecule each base position in the plurality of base positions that satisfies a filtering criterion occurs prior to bagging the first plurality of sequence reads. In some alternative embodiments, the removing occurs after bagging the first plurality of sequence reads. In some embodiments, the removing occurs prior to transforming the first sequencing dataset into a feature tensor that represents a distribution of the plurality of features in the first sequencing dataset.

Featurizing Read Bags.

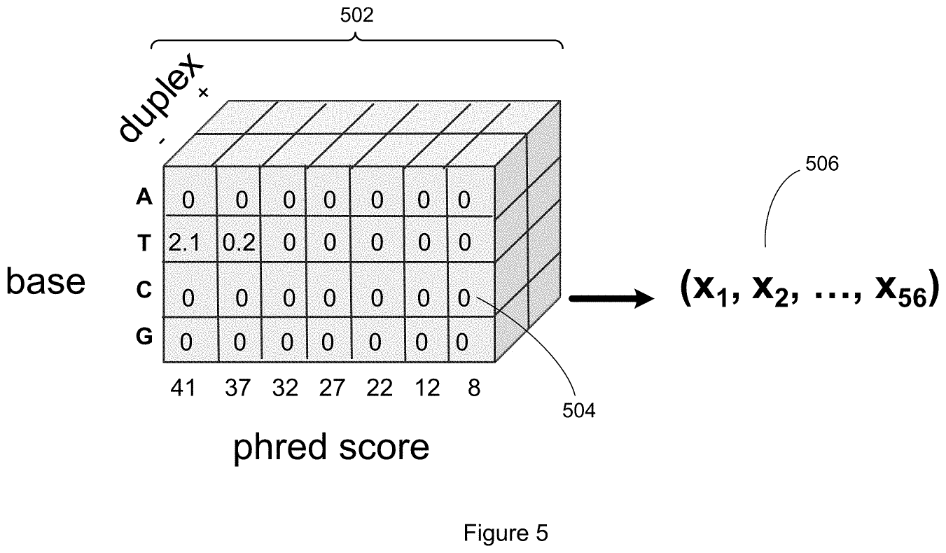

Blocks 208-212. Referring to block 208 of FIG. 2A, the method continues by transforming the first sequencing dataset into a feature tensor 128 that represents a distribution of the plurality of features in the first sequencing dataset. FIG. 5 illustrates an example multidimensional array 502 that is converted into feature tensor 506. In the example array 502, one or more values 504 are provided for each feature (e.g., dimension). In other words, each feature is assigned a dimension in the tensor. For ease of visualization, only three dimensions are drawn in FIG. 5: nucleic acid base class, phred quality score, and duplex (e.g., forward or reverse strand). However, in some embodiments, one or more other dimensions (e.g., trinucleotide context or any other feature from the group mentioned herein) are included as additional dimensions in the feature tensor. In some embodiments, all of the values for the two or more dimensions of the feature tensor can be compressed into one score. In some embodiments, as illustrated in FIG. 5, these multiple dimensions are collapsed into a one-dimensional feature tensor (e.g., 506).

Referring to block 210, in some embodiments, the transformation of the first sequencing dataset into the feature tensor comprises converting the features in the first sequencing dataset into a multidimensional array and flattening (e.g., compressing) the multidimensional array into a one-dimensional vector. In some embodiments, the number of elements in this vector is determined by the product of the selected features from the sequencing dataset (e.g., if the selected features are base reads and base read quality scores, then the number of elements in the feature tensor is determined from the number of possible base classes (e.g., 4)—A, T, C, G, or A, U, C, G—multiplied by the number of possible quality scores (e.g., 3 or 7) would result in 28 elements in the resulting vector). An example dataset is provided in Table 3 below.

TABLE 3
Example sequencing dataset

| Base | | | Strand | | |
|---|---|---|---|---|---|
| position | Base reads | Phred scores | Identifier | Overlap | End Distance |
| 1 | T, T, T, T, T | 41, 41, 37, 41, 41 | 0, 1, 1, 1, 0 | 1, 1, 1, 1, 1 | 30, 30, 30, 30, 30 |
| 2 | A, A, A, A | 37, 32, 41, 41 | 0, 0, 1, 0 | 0, 0, 0, 0 | 11, 11, 11, 11 |
| 3 | G, G, G, G | 41, 32, 41, 22 | 1, 1, 1, 1 | 0, 0, 0, 0 | 55, 55, 55, 55 |
| . | . | . | . | . | . |
| . | . | . | . | . | . |
| . | . | . | . | . | . |

The sequencing dataset in Table 3 can be converted into a multidimensional array such as array 502 shown in FIG. 5. Each column (except for the base position) comprises a potential feature (e.g., base reads, phred—base read quality scores, duplex status, overlap status, and end distance). Additional features that are not shown here can also be included (e.g., a terminal base indication, a trinucleotide context, ambiguous strand count, mapping quality, distance of the base position from a variant such as a homopolymer, insertion, or deletion, a duplex interaction score, and a trinucleotide confidence score). At least two features must be selected to proceed with determining a consensus base.

The conversion into the multidimensional array 502 includes some initial flattening of the initial sequencing dataset values for each feature. For example, for base position 2 in Table 3, the array values for phred scores can be determined for each possible phred score (e.g., 41, 37, 32, 27, 22, 12, and 8). For base position 2, the phred scores for the plurality of base reads are 41, 41, 37, and 32. There are no base reads with phred scores of 27 or lower. There is one base read with each of the phred scores 37 and 32, and there are two base reads with an associated phred score of 41. Hence, as explained below with regard to discounted values, a value of 1 is determined from the sequencing dataset and assigned to each the phred scores 37 and 32 (e.g., one base read is associated with each of those phred scores). However, phred score 41 is assigned a discounted value of 1.5, which indicates that two base reads have a phred quality score of 41. In some embodiments, each first base read with each respective phred quality score is assigned a discounted value of 1. In some embodiments, each second base read with each respective phred quality score is assigned a discounted value of ½ (e.g., 1/(the rank of the respective base call), where the rank is 2). In some embodiments, each third base read with each respective phred quality score is assigned a discounted value of ⅓ (e.g., 1/[the rank of the respective base call], where the rank is 3). This discounting continues for each additional base read for each phred quality score and serves to weight all of the base reads. The initial flattening into a multidimensional array proceeds as described above for each feature that is considered.

In some alternative embodiments, the discounted value for each base read is determined based on the ranking of each respective base read for the plurality of phred quality scores (e.g., where the ranking and corresponding discounted value assignment is not reset for each respective phred quality score). Thus, referring to base position 2 of Table 3, for the plurality of base reads with corresponding phred scores 41, 41, 37, and 32, the discounted values assigned to each base read would be 1, ½, ⅓, and ¼, respectively. The discounted values for each phred score, therefore, would be 1.5 for phred score 41, 0.33 for phred score 37, and 0.25 for phred score 32. FIG. 5 illustrates one such example of the discounted value assignment for base position 1 of Table 3, for the plurality of base reads with corresponding phred scores 41, 41, 41, 41, and 37. The discounted value for phred score 41 here is, for example, 2.4 (1+½+⅓+¼), and the discounted value for phred score 37 is 0.2 (⅕), corresponding to ranks 1, 2, 3, 4, and 5.

After a sequencing dataset is converted into a multidimensional array, the multidimensional array is then converted into a feature tensor (e.g., feature tensor 506 in FIG. 5). This follows a similar process to that described above with regard to compressing multiple values from the sequencing dataset for the multidimensional array.

In some embodiments, transforming of the sequencing dataset into the feature tensor further comprises representing values associated with the additional features at one or more elements appended to the feature tensor.

Referring to block 212, in some embodiments, the feature tensor represents a quantified distribution of the features in the first sequencing dataset. In some such embodiments, the quantified distribution is determined by: sorting each base read in the first plurality of base reads by rank; generating a value for each base read in accordance with its respective rank; and representing the value at each base read in the feature tensor. In some embodiments, the features for each base read comprise a respective nucleotide base and a respective read quality score. In such embodiments, the quantified distribution comprises a discounted (e.g., down-weighted) distribution that is determined by: i) ranking the first plurality of base reads by order of decreasing quality scores such that each base read in the first plurality of base reads has a respective quality score rank number “k”; ii) generating the value for each base read with a discounted value based on its respective quality score rank number “k”; and iii) representing the discounted value of each base read at an element in the feature tensor that coincides with a respective nucleotide base and a respective read quality score of the base read.

In such embodiments, each element coinciding with a single base read is assigned the discounted value of the single base read. In such embodiments, each element coinciding with a plurality of base reads is assigned a sum of the discounted values of the plurality of base reads (e.g., a sum of each respective discounted value of each base read in the plurality of base reads). In such embodiments, each element coinciding with no base reads is assigned a zero or null value. In some embodiments, the discounted value is determined from a function of the respective sequencing quality score rank of the respective base read. In some embodiments, the function is 1/k. In some embodiments, the function is 1/log(k).

In some embodiments, ranking occurs separately for each base identity in a set of base identities (e.g., A, C, G, and T or U) for a respective base position. The first value for each base identity is 1 count. Each additional count is determined as an additional discounted count value. In some embodiments, the discounted value is based on the respective rank number “k” of each base read and is predetermined to be 1/k or 1/log(k). In some embodiments, the discounted count value is learned from the data itself. For example, consider the case where the following pattern of base calls and quality scores is observed for a respective base position: (A, Q=40), (A, Q=36), and (C, Q=32). Then the A and C base identities are assigned 1.5 counts (e.g., 1+½, where the denominator is k) and 1 count, respectively. See, for example, the abovementioned descriptions of Table 3 and FIG. 5 for various implementations of assigning discounted values.

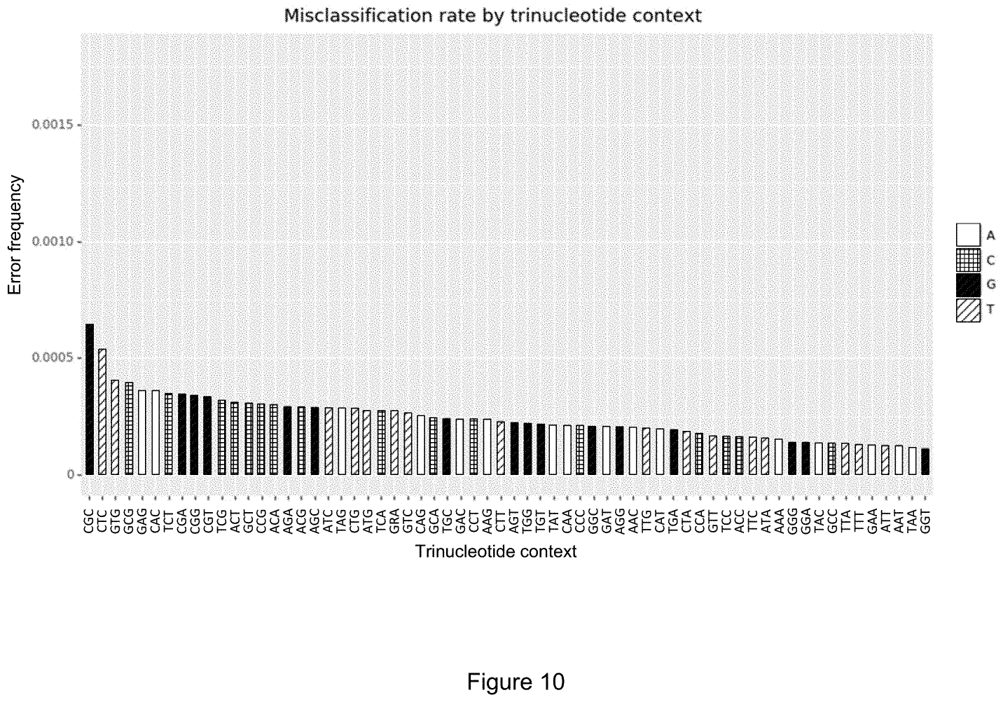

As illustrated in FIG. 10, there are different error rates for each trinucleotide context. The x-axis of FIG. 10 shows each possible trinucleotide using the set of base classes including A, T, G, and C. Similar trinucleotide permutations exist for the set of base classes that includes A, U, G, and C. In FIG. 10, the correct (e.g., known from the reference genome) base call corresponds to the middle nucleotide, and the error rate refers to the frequency of misclassification of the middle nucleotide (e.g., the base position being analyzed). In some embodiments, including additional information, such as the base reads of surrounding nucleotides, for each base read (e.g., as a feature) improves the model performance in determining the consensus base call for the base position.

Analyzing error rates across both trinucleotide and dinucleotide contexts provides useful information regarding assay noise/errors introduced in library prep and prior to bag collapsing. In particular, in some embodiments, in trinucleotide contexts, there are error peaks in CpG contexts. In some embodiments, using different library prep methods reduce CpG errors prior to sequencing, and also reduce collapser error across certain CpG trinucleotide contexts.

Predicting Base Calls Using Classifiers.

Blocks 214-224. Referring to block 24 of FIG. 2B, the method continues by assessing the feature tensor with a classifier 140 to determine the consensus base call 130 for the first base position, where the consensus base call comprises a predicted nucleotide base.

As discussed above with regard to FIG. 4, the one or more sequence reads (each with an associated nucleic acid base read for a respective base position), are collapsed into one consensus base call for the respective base position (e.g., features of the base reads 402 are analyzed to produce one consensus base call 404).

In some embodiments, the method is repeated one or more times (e.g., for determining consensus base calls for multiple base positions, for instance as part of a gene, a genomic loci, a genomic region of interest, etc.). For example, in some embodiments, the method further comprises d) repeating the obtaining (a), transforming (b), and assessing (c) for at least 10 base positions, at least 100 base positions, at least 1000 base positions, or at least 10,000 base positions. In some embodiments, the method is repeated (d) for between 1 and 100 base positions, between 1 and 1000 base positions, between 1 and 5000 base positions, between 500 and 5000 base positions, between 1000 and 5000 base positions, or between 2500 and 10,000 base positions.

Referring to block 216, in some embodiments, assessing the feature tensor with the classifier comprises computing class probabilities for the based position based on the feature tensor. In such embodiments, the elements of the feature tensor serve as inputs to the classifier and, responsive to such input, the classifier determines class probabilities for the subject base position.

In some embodiments, assessing the feature tensor with the classifier further comprises determining the consensus base call based on a highest-class probability among the class probabilities. In some embodiments, a quality score for the consensus base call is determined from the class probabilities (e.g., as described with regard to blocks 208-212).

Referring to block 218, in some embodiments, the classifier is trained with a regression model to derive model coefficients for predicting a nucleotide base and a model quality score from the features represented in the feature tensor. In some embodiments, the classifier is trained with a penalized logistic regression model. In some embodiments, the classifier is trained with a multinomial logistic regression model with L1 or L2 regularization. In some embodiments, principal component analysis of training data is used to visualize then feature space. In some such embodiments, selection of the regression model used to derive model coefficients is based at least in part on the distribution of training data in PCA feature space. In some embodiments, the regression model used to derive model coefficients is selected from the set of a linear regression model, a logistic regression model, a polynomial regression model, or other type of regression model.

In some embodiments, the classifier is a supervised learning algorithm. Nonlimiting examples of supervised learning algorithms include, but are not limited to, multivariate logistic regression models, a neural networks, convolutional neural networks (deep learning algorithm), support vector machines (SVM), Naïve Bayes algorithms, a nearest neighbor algorithms, random forest algorithms, decision tree algorithms, boosted tree algorithms, regression algorithms, logistic regression algorithms, multi-category logistic regression algorithms, linear discriminant analysis algorithms, and supervised clustering models.

Neural Networks. Neural network algorithms, also known as artificial neural networks (ANNs), and further including convolutional neural network algorithms (deep learning algorithms), are disclosed in Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Neural networks are machine learning algorithms that may be trained to map an input data set to an output data set where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) is a neural network comprising a plurality of hidden layers, e.g., two or more hidden layers. Each layer of the neural network comprises a number of nodes (or “neurons”). A node receives input that comes either directly from the input data (e.g., copy number values) or the output of nodes in previous layers, and performs a specific operation, e.g., a summation operation. In some embodiments, a connection from an input to a node is associated with a weight (or weighting factor). In some embodiments, the node may sum up the products of all pairs of inputs, xᵢ, and their associated weights. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, sinusoid, sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) (e.g., a determination of cancer condition) that the ANN computes are consistent with the examples included in the training data set. The parameters may be obtained from a back propagation neural network training process that may or may not be performed using the same computer system hardware as that used for performing the cell-based sensor signal processing methods disclosed herein.

Any of a variety of neural networks known to those of skill in the art may be suitable for use in the present disclosure. Examples include, but are not limited to, feedforward neural networks, radial basis function networks, recurrent neural networks, convolutional neural networks, and the like. In some embodiments, the disclosed classifier is a pre-trained ANN or deep learning architecture.

In general, the number of nodes used in the input layer of the ANN or DNN may range from about 10 to about 100,000 nodes. In some embodiments, the number of nodes used in the input layer is at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, or at least 100,000. In some embodiments, the number of nodes used in the input layer may be at most 100,000, at most 90,000, at most 80,000, at most 70,000, at most 60,000, at most 50,000, at most 40,000, at most 30,000, at most 20,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes.

In some embodiments, the total number of layers used in the ANN or DNN (including input and output layers) ranges from about 3 to about 20. In some embodiments, the total number of layers is at least 3, at least 4, at least 5, at least 10, at least 15, or at least 20. In some embodiments, the total number of layers is at most 20, at most 15, at most 10, at most 5, at most 4, or at most 3. Those of skill in the art will recognize that the total number of layers used in the ANN may have any value within this range, for example, 8 layers.

In some embodiments, the total number of learnable or trainable parameters, e.g., weighting factors, biases, or threshold values, used in the ANN or DNN ranges from about 1 to about 10,000. In some embodiments, the total number of learnable parameters is at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters is any number less than 100, any number between 100 and 10,000, or a number greater than 10,000. In some embodiments, the total number of learnable parameters is at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Those of skill in the art will recognize that the total number of learnable parameters used may have any value within this range, for example, about 2,200 parameters.

SVMs. SVM algorithms that can be used in the present disclosure are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary-labeled data training set with a hyper-plane that is maximally distant from the labeled data. For cases in which no linear separation is possible, SVMs can work in combination with the technique of “kernels”, which automatically realizes a non-linear mapping to a feature space. The hyper-plane found by the SVM in feature space corresponds to a non-linear decision boundary in the input space.

Naïve Bayes algorithms. Naive Bayes classifiers are a family of “probabilistic classifiers” based on applying Bayes' theorem with strong (naïve) independence assumptions between the features. In some embodiments, they are coupled with Kernel density estimation. See, Hastie, Trevor, 2001, The elements of statistical learning: data mining, inference, and prediction, Tibshirani, Robert, Friedman, J. H. (Jerome H.), New York: Springer, which is hereby incorporated by reference.

Nearest neighbor algorithms. Nearest neighbor classifiers are memory-based and require no classifier to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−x₍₀₎∥

In some embodiments, when the nearest neighbor algorithm is used, the bin values for the training set are standardized to have mean zero and variance 1. In some embodiments, the nearest neighbor analysis is refined to address issues of unequal class priors, differential misclassification costs, and feature selection. Many of these refinements involve some form of weighted voting for the neighbors. For more disclosure on nearest neighbor analysis, see Duda, Pattern Classification, Second Edition, 2001, John Wiley & Sons, Inc.; and Hastie, 2001, The Elements of Statistical Learning, Springer, New York, each of which is hereby incorporated by reference in its entirety.

Random forest, Decision Tree, and boosted tree algorithms. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Regression, logistic regression, and multi-category logistic regression. The regression algorithm can be any type of regression. For example, in some embodiments, the regression algorithm is logistic regression. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the regression algorithm is logistic regression with lasso, L2 or elastic net regularization. In some embodiments, features that have a corresponding regression coefficient that fails to satisfy a threshold value are pruned (removed from) consideration. In some embodiments, this threshold value is zero. In some embodiments, a generalization of the logistic regression model that handles multicategory responses serves as the classifier. A number of such multi-category logit models described in Agresti, An Introduction to Categorical Data Analysis, 1996, John Wiley & Sons, Inc., New York, Chapter 8, hereby incorporated by reference in its entirety. In some embodiments the regression is linear regression. See, for example, Edwards, 1984, An Introduction to Linear Regression and Correlation, Second Edition, W.H. Freeman and Company, New York, which is hereby incorporated by reference.

Linear discriminant analysis algorithms. Linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. The resulting combination is used as the classifier (linear classifier) in some embodiments of the present disclosure. See, McLachlan, 2004, Discriminant Analysis and Statistical Pattern Recognition, Wiley Interscience, ISBN 978-0-471-69115-0. MR 1190469, which is hereby incorporated by reference.

Ensembles of classifiers and boosting. In some embodiments, an ensemble (two or more) of classifiers is used. In some embodiments, a boosting technique such as AdaBoost is used in conjunction with many other types of learning algorithms in the present disclosure. In this approach, the output of any of the classifiers disclosed herein, or their equivalents, is combined into a weighted sum that represents the final output of the boosted classifier. See, Freund, 1997, “A decision-theoretic generalization of on-line learning and an application to boosting,” Journal of Computer and System Sciences 55, p. 119, which is hereby incorporated by reference.

Clustering. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

More recently, Duda et al., Pattern Classification, 2^{nd} edition, John Wiley & Sons, Inc., New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J., each of which is hereby incorporated by reference. Particular exemplary clustering techniques that can be used in the present disclosure include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. Such clustering can be on a set of features (or the principal components derived from the set of first features). In some embodiments, the clustering comprises unsupervised clustering where no preconceived notion of what clusters should form when the training set is clustered are imposed.

In some embodiments, somatic and germline mutations are removed from the training data. In alternative embodiments, somatic and germline mutations are included in the training data.

In some embodiments, the training data comprises a plurality of base reads for base positions from stitched regions. In some embodiments, the training data comprises a plurality of base reads for base positions from non-stitched regions. In some embodiments, the training data comprises a plurality of base reads from whole genome bisulfite sequencing or from bisulfite sequencing (e.g., targeted bisulfite sequencing or reduced representation bisulfite sequencing). In some embodiments, the training data comprises base positions with low depth bag counts. In some embodiments, the training data comprises base positions that are terminal bases (e.g., edge base positions). In some embodiments, the training data comprises base positions that are terminal bases and base positions that are within a predetermined distance from an end of a respective sequence read. In some embodiments, the predetermined distance from an end of a respective sequence read is less than 10 bases from an end, less than 9 bases from an end, less than 8 bases from an end, less than 7 bases from an end, less than 6 bases from an end, less than 6 bases from an end, less than 5 bases from an end, less than 4 bases from an end, less than 3 bases from an end, or less than 2 bases from an end.

In some embodiments, the training sets (used to train the classifier) and testing sets (used to assess the performance of the classifier) comprise genomic DNA (gDNA) data from a reference genome. In some embodiments, the reference genome is NA12878 platinum. In some embodiments, SNP positions are removed from the reference genome, such that each reference base label in both the training and testing sets represents a true base at a given position. In some embodiments, the training and testing sets comprise cell-free DNA (cfDNA) data from a healthy control group. In such embodiments, base positions with two or more sequence reads that support an alternate allele are removed.

In some such embodiments, the reference genome is obtained using a sequencing method other than the sequencing method used to obtained the sequencing dataset (e.g., where the sequencing dataset for the target nucleic acid molecule is obtained using a methylation sequencing, and the reference genome is obtained using a targeted sequencing).

In some embodiments, the training comprises n-fold cross-validation (e.g., to optimize model parameters such as regularization) with the multinomial logistic regression of the features selected from the group. In some embodiments, the training comprises 10-fold cross-validation with the multinomial logistic regression of the features selected from the group.

In some embodiments, the training set has 10 million examples of bag pileups sampled across randomly selected genomic positions. In some embodiments, the testing set has 10 million examples of bag pileups sampled across randomly selected genomic positions. In some embodiments, the training set has at least 100,000 examples, at least 250,000 examples, at least 500,000 examples, at least 1 million examples, at least 2 million examples, at least 3 million examples, at least 4 million examples, at least 5 million examples, at least 6 million examples, at least 7 million examples, at least 8 million examples, at least 9 million examples, at least 10 million examples, at least 11 million examples, at least 12 million examples, at least 13 million examples, at least 14 million examples, or at least 15 million examples of bag pileups sampled across randomly selected genomic positions. In some embodiments, the testing set has at least 100,000 examples, at least 250,000 examples, at least 500,000 examples, at least 1 million examples, at least 2 million examples, at least 3 million examples, at least 4 million examples, at least 5 million examples, at least 6 million examples, at least 7 million examples, at least 8 million examples, at least 9 million examples, at least 10 million examples, at least 11 million examples, at least 12 million examples, at least 13 million examples, at least 14 million examples, or at least 15 million examples of bag pileups sampled across randomly selected genomic positions.

In some embodiments, the set of model coefficients is used for determining consensus base calls for sequence reads from a first assay type. In some embodiments, retraining the classifier comprises computing a second set of model coefficients for determining consensus base calls for sequence reads from a second assay type distinct from the first assay type)

Referring to block 220, in some embodiments, the classifier comprises a multinomial logistic regression model and a set of model coefficients learned during training of the classifier. In some embodiments, the classifier comprises a random forest. In some embodiments, the classifier comprises a neural network algorithm, a support vector machine algorithm, a Naive Bayes algorithm, a nearest-neighbor algorithm, or a boosted trees algorithm.

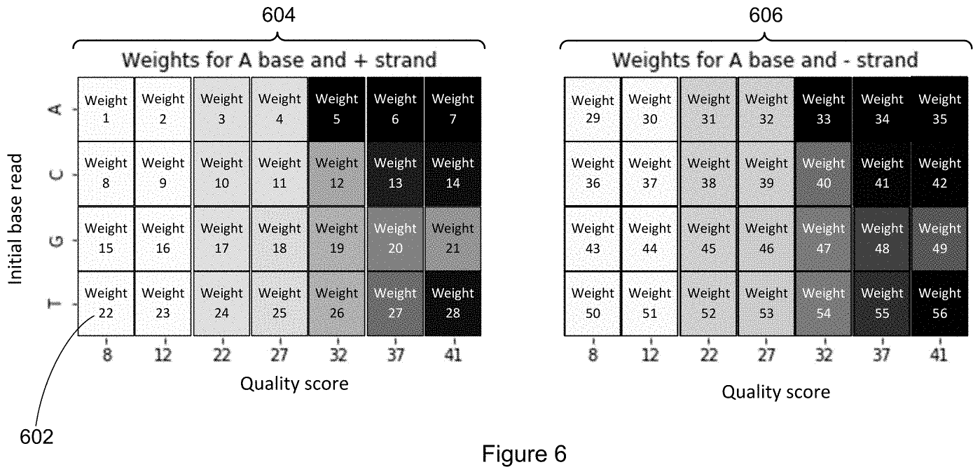

In some embodiments, one or more model coefficients (e.g., weights 1-56) are used to determine a probability for a base read. FIG. 6 illustrates example weights used for base A, where panel 602 contains weights associated with base calls for a forward strand, and panel 604 corresponds to weights associated with base calls for a reverse strand. In some embodiments, weights (e.g., model coefficients) are determined for each nucleotide base in a set of classes (e.g., a set of base types) that include at least four classes (e.g., base types or base identities) selected from a group comprising adenine (“A”), guanine (“G”), thymine (“T”), cytosine (“C”), and uracil (“U”). In some embodiments, the set of classes comprises A, T, G, and C. In some embodiments, the set of classes comprises A, U, G, and C. In some embodiments, model coefficients weights are broken down by forward and reverse strand (e.g., panels 604 and 606, respectively). In some embodiments, weights for each nucleotide base are not separated into weights for forward and reverse strands. In some embodiments, distinct model coefficients are provided based on a respective quality score (e.g., a phred quality score) of each base read (e.g., 8-41 in the x-axes here). In some embodiments, model coefficients for each base read increase along with phred quality score. In some embodiments, distinct model coefficients are determined for each trinucleotide base context (e.g., as described above with regard to blocks 204-206).

Referring to block 222, in some embodiments, the at least two features for determining the consensus base call are selected at least in part based on principal component analysis of misclassified examples of training data (e.g., by using PCA of misclassified examples). In some embodiments, the at least two features for determining the consensus base call are selected using a feature selection method of misclassified training data examples.

In some such embodiments, one or more features can be selected based on analysis of the distribution of training data (e.g., the data distributed in PCA feature space). In some embodiments, principal component analysis is used to reduce the feature vector (e.g., convert the original feature vector into a smaller, transformed feature vector with fewer features). In some embodiments, reducing the feature vector comprises recasting the original feature vector onto principal components axes, thereby providing a transformed feature vector. In some embodiments, this transformed feature vector determines the features used for a predictive model (e.g., the classifier).

In some embodiments, the regression model itself is used to select one or more features. Methods of data visualization (e.g., to observe data distribution in feature space) compatible with blocks 222 further include t-SNE (t-Distributed Stochastic Neighbor Embedding) and UMAP (Uniform Manifold Approximation and Projection). See e.g., van der Maaten et al., 2008, J Machine Learning Res 9, 2579-2605 and McInnes et al., 2018, arXiv:1802.03426, respectively). These methods are particularly useful for visualizing high-dimensionality data and identifying data clusters, and, in some embodiments, features.

In some embodiments, feature selection methods suitable for use with block 222 include L1 penalization, forward/backward selection, and random forest analysis. See e.g., Destrero et al 2008 Comp Management Sci 6(1), 25-40; Mao IEEE Transactions on Sys 34(1), 629-634; and Li et al., 2011 BMC Bioinformatics 12, 450. In some embodiments, feature selection methods provide an indication of feature importance (e.g., a quantitative measurement of the influence of each feature on the distribution of the data). In some embodiments, a higher quantitative measurement indicates a more influential feature. In some embodiments, a set of features is selected based at least in part on the quantitative measurement of feature influence (e.g., one or more features with an influence measurement above a predefined threshold value are selected). In some embodiments, the set of features comprises at least 1 feature, at least 2 features, at least 3 features, at least 4 features, at least 5 features, at least 6 features, at least 7 features, at least 8 features, at least 9 features, or at least 10 features.

Calculating and Recalibrating Quality Scores.

Referring to block 224, in some embodiments, the classifier determines a model quality score for the consensus base call. In some embodiments, the model quality score is of the form:

Q=−10·(Pₑᵣᵣₒᵣ)

In such embodiments, Pₑᵣᵣₒᵣ is a probability that the predicted nucleotide base is incorrect and is of the form:

Pₑᵣᵣₒᵣ=1−P_{class}

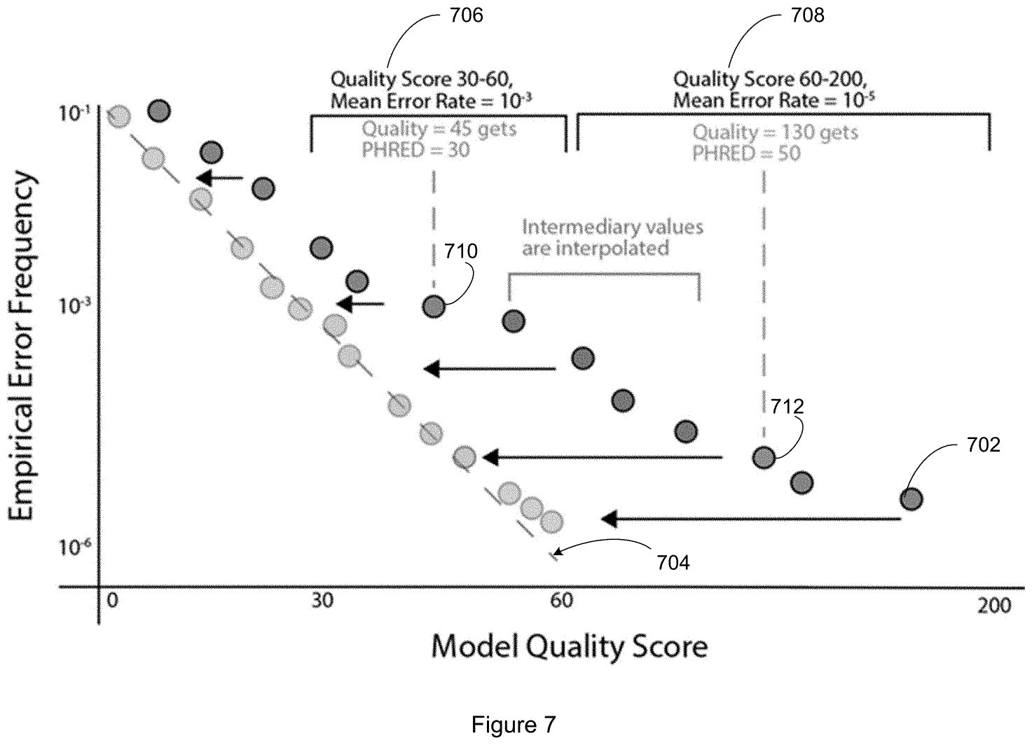

In such embodiments, P_{class} is based on a class probability of the predicted nucleotide base computed by the classifier. In some embodiments, Q is determined based on a recalibration of the model quality score performed after classification (e.g., such as the recalibration illustrated in FIG. 7 and described below). In some embodiments, the method further comprises recalibrating the model score based on an empirical error rate. In some embodiments, this recalibration further provides Q.

In some embodiments, the recalibrated score improves downstream variant calling with low read support and/or facilitates variant calls in samples with low tumor fraction. In some embodiments, the empirical error rate is based on NA12878 cell line training data used to train a gDNA reference model. In some embodiments, the empirical error rate is based on healthy donor cfDNA that is used to train cfDNA reference model. In such embodiments, filtering is implemented to remove any potential variant sites (e.g., sites that have a higher number of bags supporting a SNP or other variant than a reference sequence). In some embodiments, the empirical error rate is a misclassification rate of the classifier on the training data.

In some embodiments, recalibrating the model quality score comprises i) identifying a quality bin corresponding to the model quality score, and ii) interpolating the model quality score based on an empirical error rate at the quality bin to generate a recalibrated model quality score. In some embodiments, the quality bin is one of a plurality of quality bins defined by model quality scores obtained during training of the classifier with training data.

FIG. 7 illustrates one example of recalibration. For example, FIG. 7 illustrates a set of example model quality scores (e.g., the dark circles 702). This set of example model quality scores has been divided into two quality bins (e.g., bin 706 including those scores with a mean empirical error rate of 10⁻³, and bin 708 including those scores with a mean empirical error rate of 10⁻⁶). As illustrated by FIG. 7, the model quality scores are recalibrated (e.g., are converted into the light grey circles 704) based on which bin they are in (e.g., the magnitude of the recalibration depends on the bin). For instance, score 710 is converted from an initial model quality score of 45 to a recalibrated quality score of 30, and score 712 is converted from an initial model quality score of 130 to a recalibrated quality score of 50. Thus, model quality scores are adjusted (e.g., recalibrated) based on a combination of each model quality score and the mean empirical error rates. The resulting recalibrated model quality scores, as can be seen in FIG. 7, are more regular (e.g., are aligned in line 704) than the non-calibrated scores such as 702.

In some embodiments, the computed model quality score comprises too high of a quality score (e.g., a quality score suggests a higher confidence than is supported by the data). In some embodiments, the recalibration is performed by linear interpolation or non-linear (spline based) interpolation. For examples of spline-based interpolation equations, see Schoenberg 1946 Quarterly of Applied Mathematics 4(2), 45-99 and Schoenberg 1946 Quarterly of Applied Mathematics 4(2), 112-141. North and Livingstone 2013 provide a comparison of linear and spline-based interpolation (Limnol Oceanog: Methods 11, 213-224). However, the methods referenced here serve merely as examples and are not intended to limit methods performed as described herein. Other interpolation methods may be used to recalibrate quality scores.

In some embodiments, recalibration of model scores is performed using the combined error rate of all possible base identities (e.g., A, C, G, T and/or U). In some embodiments, recalibration of model scores is performed on a per-base basis (e.g., where each base identity is associated with a different error rate). In some such embodiments, the recalibration is performed using a first subset of possible base identities (e.g., A, G, and T) and the error rate of each remaining base identity (e.g., C) is estimated as an average of known error rates for the first subset of base identities, and/or by interpolating the raw error rate of the respective remaining base identity into the recalibrated model.

Generating Consensus Sequences.

Block 226. Referring to block 226, in some embodiments, the method further comprises determining a plurality of consensus base calls for a plurality of base positions of the target nucleic acid molecule to generate a consensus sequence. In some embodiments, the plurality of base positions comprises 5 or more consecutive base positions, 10 or more consecutive base positions, 20 or more consecutive base positions, 50 or more consecutive base positions, 100 or more consecutive base positions, 200 or more consecutive base positions, 300 or more consecutive base positions, 400 or more consecutive base positions, 500 or more consecutive base positions, or 1000 or more consecutive base positions. In some embodiments, determining a plurality of consensus base calls for a plurality of base positions comprises repeating the method for determining a respective consensus base call for each base position in the plurality of base positions. In some embodiments, determining a plurality of consensus base calls for a plurality of base positions comprises performing in parallel (e.g., via parallel processing) the method for determining a respective consensus base call for each base position in the plurality of base positions.

In some embodiments, the method further comprises determining a plurality of consensus base calls for a plurality of base positions of each target nucleic acid molecule in a plurality of target nucleic acid molecules, thus generating a plurality of consensus sequences. For example, in some embodiments, the method comprises obtaining a plurality of sequence read pileups (e.g., bags), each pileup comprising a plurality of sequence reads corresponding to a different portion or fragment of a reference genome (e.g., a plurality of cfDNA molecules obtained from a biological sample). The methods and/or embodiments described herein can be performed for each base position in the plurality of base positions in each respective bag in the plurality of bags. Thus, each respective consensus sequence in the plurality of consensus sequences corresponds to each respective bag in the plurality of bags. In some such embodiments, the plurality of consensus sequences is aligned (e.g., mapped) to a reference genome.

### EXAMPLES

### Example 1

### The Cancer Genome Atlas (TCGA) Samples

In some embodiments, genotypic information is obtained using data from the Cancer Genome Atlas (TCGA) cancer genomics program that is led by the National Cancer Institute and the National Human Genome Research Institute. The TCGA dataset comprises, among other information, gene expression profiles from a large number of human cancer samples. The information is obtained using high-throughput platforms including gene expression mutation, copy number, methylation, etc. Briefly, the TCGA dataset is a publicly available dataset comprising more than two petabytes of genomic data for over 11,000 cancer patients, including clinical information about the cancer patients, metadata about the samples (e.g., the weight of a sample portion, etc.) collected from such patients, histopathology slide images from sample portions, and molecular information derived from the samples (e.g., mRNA/miRNA expression, protein expression, copy number, etc.). The TCGA dataset includes data on 33 different cancers: breast (breast ductal carcinoma, bread lobular carcinoma) central nervous system (glioblastoma multiforme, lower grade glioma), endocrine (adrenocortical carcinoma, papillary thyroid carcinoma, paraganglioma, and pheochromocytoma), gastrointestinal (cholangiocarcinoma, colorectal adenocarcinoma, esophageal cancer, liver hepatocellular carcinoma, pancreatic ductal adenocarcinoma, and stomach cancer), gynecologic (cervical cancer, ovarian serous cystadenocarcinoma, uterine carcinosarcoma, and uterine corpus endometrial carcinoma), head and neck (head and neck squamous cell carcinoma, uveal melanoma), hematologic (acute myeloid leukemia, Thymoma), skin (cutaneous melanoma), soft tissue (sarcoma), thoracic (lung adenocarcinoma, lung squamous cell carcinoma, and mesothelioma), and urologic (chromophobe renal cell carcinoma, clear cell kidney carcinoma, papillary kidney carcinoma, prostate adenocarcinoma, testicular germ cell cancer, and urothelial bladder carcinoma). See, Blum et al., 2018, “TCGA-Analyzed Tumors,” SNAPSHOT 173(2), P530, which is hereby incorporated by reference.

### Example 2

### The Circulating Cell-Free Genome Atlas Study (CCGA) Samples

Subjects from the CCGA were used in the present disclosure. The CCGA (NCT02889978) CCGA is a prospective, multi-center, observational cfDNA-based, case-control early cancer detection study that has enrolled 9,977 of 15,000 demographically-balanced participants at 141 sites study with longitudinal follow-up, designed to develop a single blood test for multiple types of cancer across stages. The CCGA study develops a plasma cell-free DNA (cfDNA)-based multi-cancer detection assay. Blood was collected from subjects with newly diagnosed therapy-naive cancer (C, case) and participants without a diagnosis of cancer (noncancer [NC], control) as defined at enrollment. This preplanned substudy included 878 cases, 580 controls, and 169 assay controls (n=1627) across twenty tumor types and all clinical stages.

All samples were analyzed by: 1) paired cfDNA and white blood cell (WBC)-targeted sequencing (60,000×, 507 gene panel); a joint caller removed WBC-derived somatic variants and residual technical noise; 2) paired cfDNA and WBC whole-genome sequencing (WGS; 35×); a novel machine learning algorithm generated cancer-related signal scores; joint analysis identified shared events; and 3) cfDNA whole-genome bisulfite sequencing (WGBS; 34×); normalized scores were generated using abnormally methylated fragments. In the targeted assay, non-tumor WBC-matched cfDNA somatic variants (SNVs/indels) accounted for 76% of all variants in NC and 65% in C. Consistent with somatic mosaicism (e.g., clonal hematopoiesis), WBC-matched variants increased with age; several were non-canonical loss-of-function mutations not previously reported. After WBC variant removal, canonical driver somatic variants were highly specific to C (e.g., in EGFR and PIK3CA, 0 NC had variants vs 11 and 30, respectively, of C). Similarly, of 8 NC with somatic copy number alterations (SCNAs) detected with WGS, four were derived from WBCs. WGBS data of the CCGA reveals informative hyper- and hypo-fragment level CpGs (1:2 ratio); a subset of which was used to calculate methylation scores. A consistent “cancer-like” signal was observed in <1% of NC participants across all assays (representing potential undiagnosed cancers). An increasing trend was observed in NC vs stages I-III vs stage IV (nonsyn, SNVs/indels per Mb [Mean±SD] NC: 1.01±0.86, stages I-III: 2.43±3.98; stage IV: 6.45±6.79; WGS score NC: 0.00±0.08, I-III: 0.27±0.98; IV: 1.95±2.33; methylation score NC: 0±0.50; I-III: 1.02±1.77; IV: 3.94±1.70). These data demonstrate the feasibility of achieving >99% specificity for invasive cancer, and support the promise of cfDNA assay for early cancer detection.

### Example 3

### Improved Performance of a Machine Learning (ML)-Based Collapser Over in House Pecan Aligner

The performance of a typical method of determining consensus bases was compared to consensus base calling as performed in accordance with embodiments described herein. There are significant with the in house Pecan aligner (and also with other methods of determining consensus base calls). This is particularly evident in the failure of the Pecan aligner to correctly identify genomic variants with low levels of base read support.

For example, FIG. 12 illustrates single nucleotide variants evaluated by applying the Pecan aligner (as described by Paten et al.) to CCGA data (as described in Example 2). As shown in FIG. 12, the Pecan aligner is unable to detect variants with only 1 base read of support, and has limited success with variants with 5 or fewer supporting base reads. In total, the Pecan aligner misidentified 41% of known single nucleotide variants (e.g., variants known from tissue sequencing) that had 3-8 supporting base reads (e.g., from cfDNA sequencing). This demonstrated a need for improved analysis of sequencing data with low levels of base reads supporting each base position.

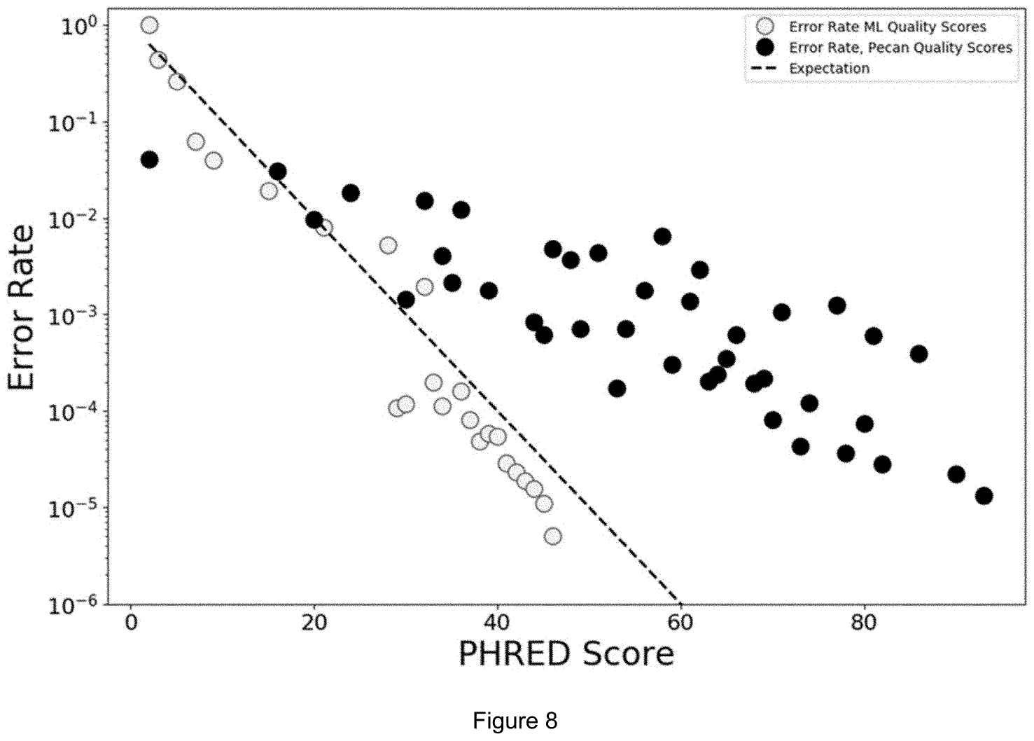

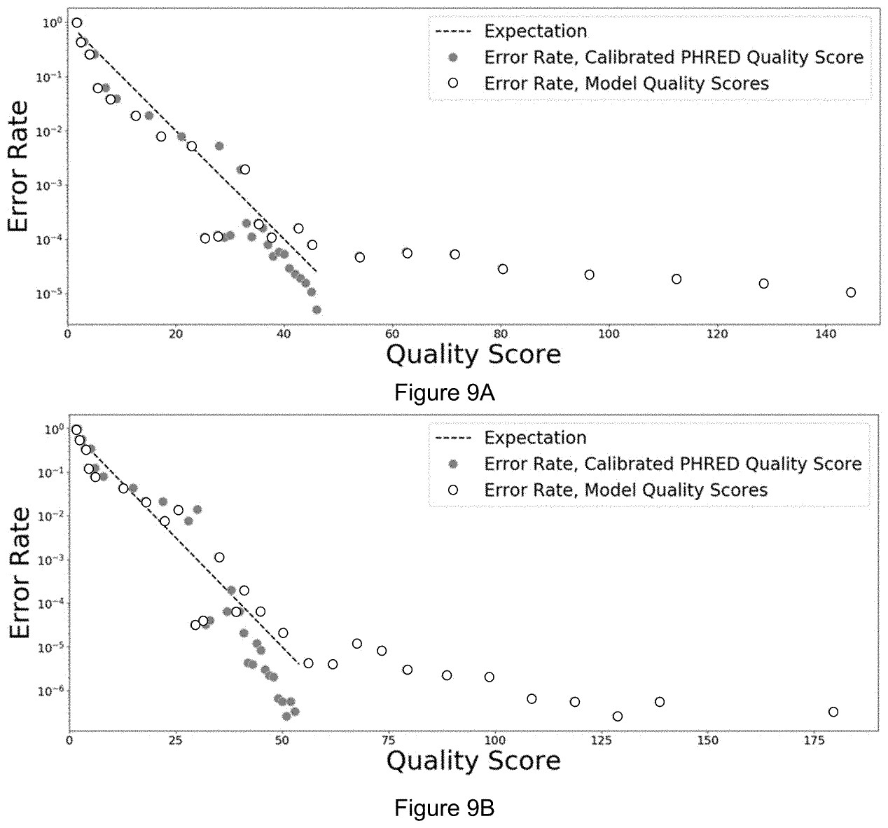

Human gDNA sequencing data from the CCGA was analyzed to compare the performance of the Pecan aligner and the currently disclosed methods (e.g., FIGS. 9B, 10). Sequencing data from the NA12878 viral genome were also used to compare model performance (FIGS. 8-9A and 11). As illustrated in FIG. 11, the error rate of the ML-based classifier (which was trained in accordance with the methods disclosed herein) is less than half that of the Pecan aligner error rate (e.g., for the highest quality scores).

As illustrated in FIG. 8, phred scores (e.g., recalibrated quality scores) determined in accordance with the ML-based classifier have associated error rates that are much closer to expected, theoretical error rates (e.g., the dashed line) than phred scores determined with the Pecan aligner. For descriptions of determining theoretical error rates see, e.g., Ruffalo et al., 2012, ECCB 28, i349-i355. For both methods, these phred scores were determined from sequencing data from the NA12878 viral genome. One reason for such differences between Pecan and classifiers trained in accordance with embodiments described herein is that, for example, Pecan uses predetermined model values to describe variation within bags (e.g., sets of base reads for a particular base position) and variation due to duplex/non-duplex sequencing (e.g., where the predetermined values are estimates of error based on control data). Thus, the ML classifier more closely represents the variation of any given set of base reads than the Pecan aligner.

The utility of recalibrated quality scores is further demonstrated in FIGS. 9A and 9B. As shown in each figure, the error rate of non-calibrated model quality scores has a long tail (e.g., open circles). However, when these model quality scores are calibrated (e.g., filled circles), they then fall along the expected, theoretical error rate (e.g., dashed line).

### Example 4

### Performance of UMI-Free ML Based Collapser

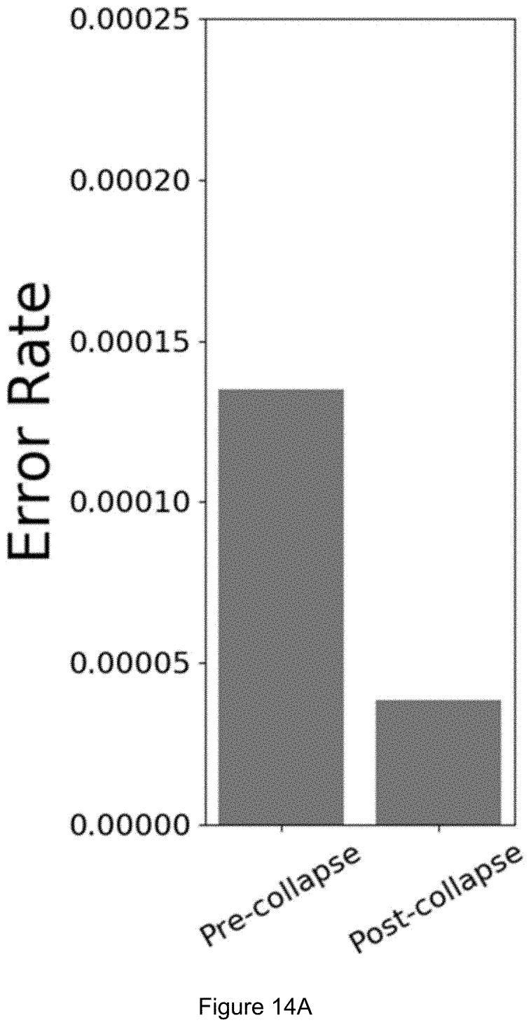

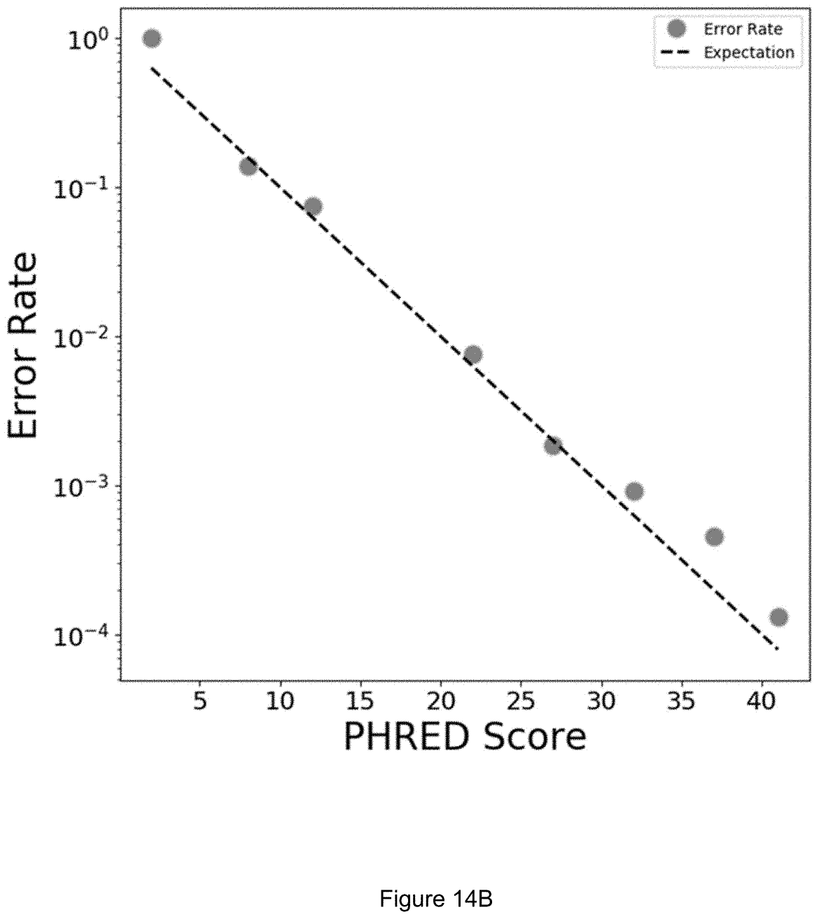

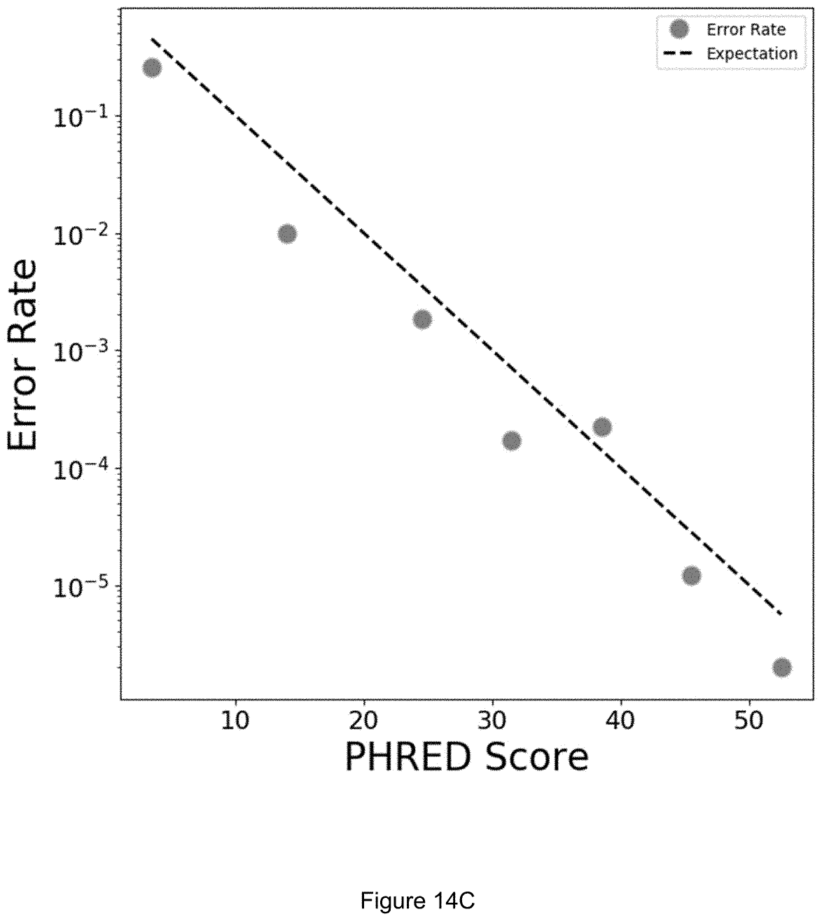

FIGS. 14A, 14B, and 14C collectively illustrate examples of error rates in determining consensus base calls using a machine learning-based collapser. Consensus base calling was performed using unique molecular identifier (UMI)-free bagging of whole genome sequencing data, in accordance with some embodiments of the present disclosure. Sequence reads were obtained from whole genome sequencing of cfDNA samples from the Circulating Cell-Free Genome Atlas study (CCGA). Sequence reads were collapsed using the ML-based collapser, in accordance with some embodiments of the present disclosure, using genomic position rather than UMI. Sequence reads were further preprocessed prior to error calculation by excluding germline variants and clonal variants (e.g., CHIP variants), as well as base positions with greater than 1 non-reference base reads (e.g., base reads differing from a reference genome obtained using targeted sequencing).

Error frequency was then evaluated on pre-collapsed and post-collapsed sequence reads for base reads at base positions present in targeted sequencing panels. For pre-collapse data, the error frequency was plotted for the highest quality bin assigned by the sequencer (e.g., phred score=41). For post-collapse data, the error frequency was plotted for all positions that were given higher phred scores by the collapser (e.g., phred score ranging from 42 to 52). As evidenced in FIG. 14A, the error rate observed during consensus base calling with the ML-based collapser (“post-collapse”) is reduced in comparison with the error rate observed during consensus base calling without the ML-based collapser (“pre-collapse”). FIGS. 14B and 14C illustrate the error rate as a function of phred score for pre-collapsed and post-collapsed base reads, respectively. The figures illustrate that collapsing base reads for consensus base calling enables higher phred scores and improved error rate.

### Example 5

### Training and Validation of ML-Based Collapser Using Targeted Methylation Data

19 matched non-cancer biological samples were obtained from human subjects enrolled in the Circulating Cell-Free Genome Atlas study (e.g., CCGA), comprising, for each sample, BAM files including a sequencing dataset obtained from targeted sequencing and a sequencing dataset obtained from targeted methylation sequencing. Targeted sequencing data was considered a “ground truth” and used to generate a reference genome for subsequent comparison of targeted methylation sequencing data. The reference genome was constructed by obtaining consensus base calls for targeted sequencing data using a ML-based collapser, where sequence reads were bagged using unique molecular identifiers (UMIs).

Targeted methylation sequencing data was prepared by sampling 38 million base reads for a training dataset and 38 million base reads for a test dataset.

To generate the ML-based collapser, a multinomial logistic regression classifier was trained using the training dataset, in accordance with some embodiments of the present disclosure. Specifically, sequence reads from the training dataset were bagged without UMIs (e.g., based on genomic position). Sequence reads were preprocessed by filtering out variant positions (e.g., comprising 2 or more alternate reads in the targeted sequencing pileup) and possible methylation positions (e.g., comprising a cytosine on the positive strand and a guanine on the negative strand), such that the true base (e.g., the reference base read) could be determined for each base position represented in each bag of methylation sequence reads.

Sequence reads from the methylation sequencing dataset were further processed after alignment to ensure that all features were evenly represented across the plurality of sequence reads, thus obtaining a consensus alignment for all sequence reads in each read bag. Specifically, consensus alignments were determined by median and/or majority votes based on CIGAR string encodings for each sequence read in the plurality of sequence reads in each bag. Median votes were used to determine the alignment start position and the leading soft-clip length across all left reads within each bag. In some cases, R1 and R2 reads of each read-pair were both considered left reads if they comprised the same pre-clipped start position. Majority votes were used to identify base positions for deletion and/or insertion. Where an insertion was designated, median votes were used to determine the length of the insertion.

Features from the training dataset were then derived from individual sequencing reads within bags, including base call identity, quality score (e.g., phred score), strand identity, read-side, and read-edge (e.g., distance from fragment end). For example, for each sequence read in the methylation sequencing dataset, each base read in the plurality of base reads was assigned a phred score of 11, 25, or 37, indicating the quality score of the base read. Additional fragment counts were discounted by sorting quality scores for each base read and weighing each additional fragment by 1/k, for each fragment rank k. Feature extraction was performed using Python scripts to evaluation features relevant for prediction. For each base read predicted by the model, a corresponding reference base read was used as the ground truth label for error evaluation and backpropagation. Features for each base read were flattened into a single-line tensor for input into the model.

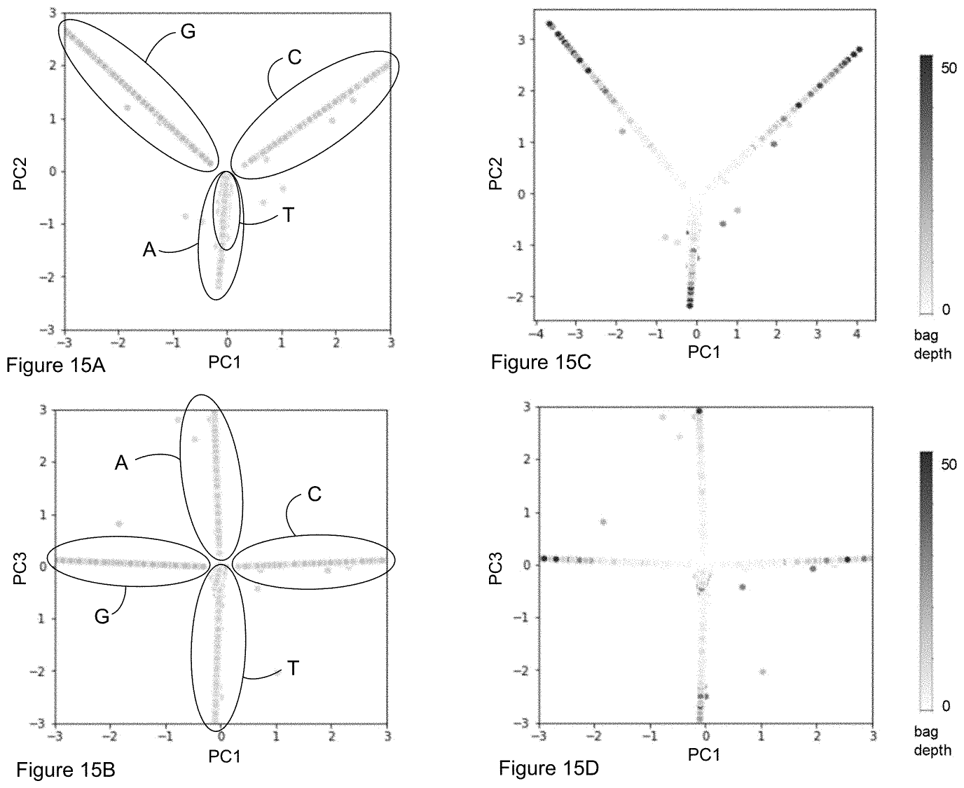

FIGS. 15A, 15B, 15C, and 15D illustrate an initial visualization of the variation between featurized bags in principal component space, and reveal that the data represented by the selected features is linearly separable. These results highlight the ability of the selected features to characterize and thus exert good discriminative power between each of the base classes A, C, G, and T, for the majority of bases. For example, FIGS. 15A and 15B illustrate that a high degree of separation between base classes can be explained by principal components 1 and 2 and principal components 1 and 3, respectively. FIGS. 15C and 15D further illustrate that the separation between classes improves with higher bag depths. Separation is more robust for positions with high bag depths (e.g., high color saturation or black) and weaker for positions with low bag depths (e.g., low color saturation or white).

Consensus base calls were computed from class probabilities reported by the multinomial logistic regression model. The probability was used to calculate quality scores for each consensus base call, using the equation QUAL=−10 log P(error). Classifier training was performed using backpropagation, resulting in a plurality of updated weights for the classifier that were then used in the trained classifier to predict the identity of collapsed bases.

The trained model was evaluated using the test dataset. Sequence reads from the test dataset were processed and bagged for feature extraction, as described above for the training dataset. Performance of the model was evaluated using class predictions obtained for the test dataset.

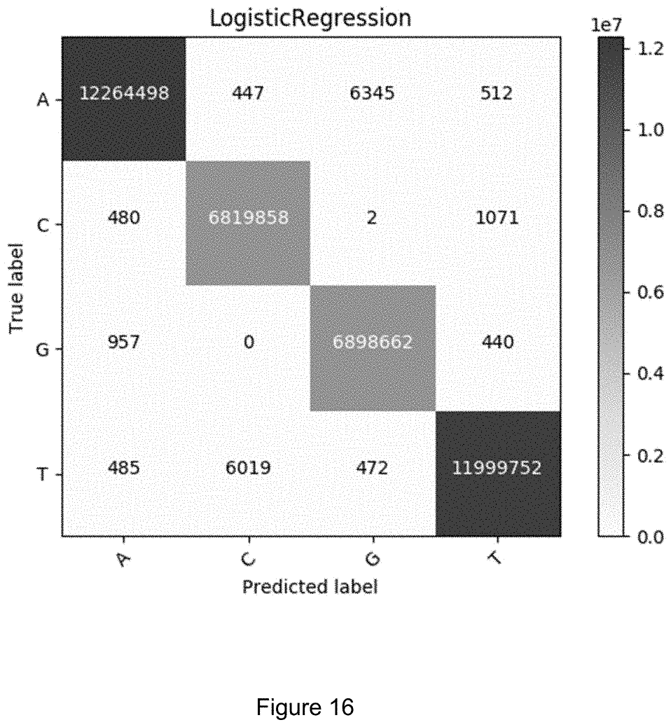

FIG. 16 illustrates the evaluation of errors using the test methylation sequencing dataset, compared with a ground truth reference genome (e.g., constructed using the targeted sequencing dataset). The heat map indicates the relative density of correctly or incorrectly predicted base reads compared to the true label. The classifier misidentified 17,230 bases out of the original 38 million base reads in the test dataset, for an error rate of ˜0.045% (e.g., ˜4.5 misclassified base reads for every 10,000 base reads). Further examination of the misidentified cases revealed that 20% of the misclassified cases had a bag depth of 1 or 2 and 80% of the misclassified cases comprised perfect evidence for the predicted base (e.g., homozygous base calls), suggesting that the majority of misclassified examples cannot be corrected.

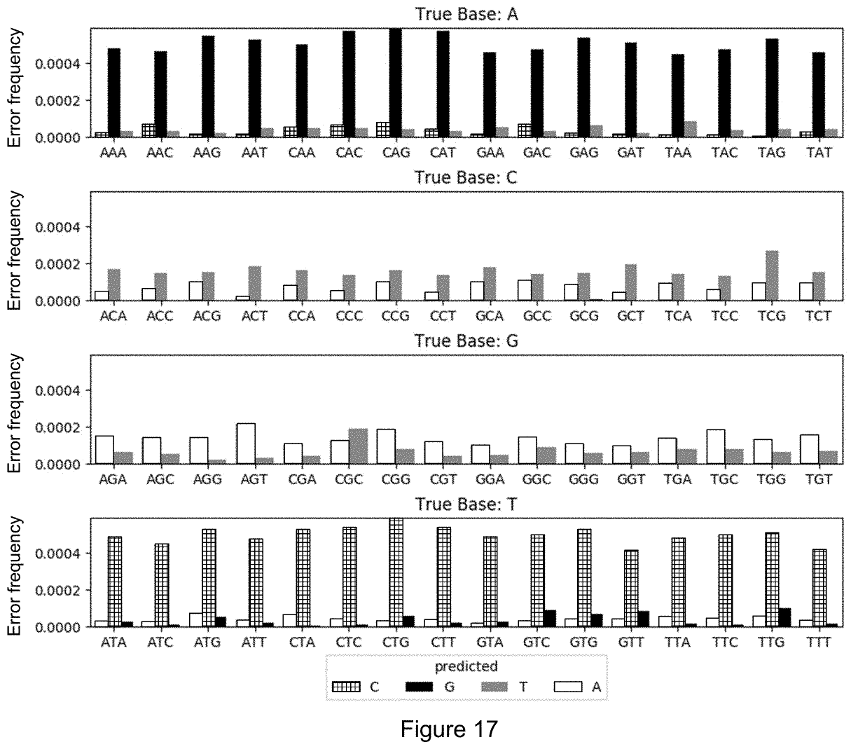

FIG. 17 illustrates the error frequency for each central base across different trinucleotide contexts. The true base label of each central base is indicated by the “True Base” heading at the top of each panel, while the trinucleotide context is indicated by the x-axis labels at the bottom of each panel. The predicted base label of each central base is indicated by the shaded bars (e.g., patterned: “C”; solid black: “G”; solid gray: “T”; solid white: “A”). Notably, although the error frequencies show a high rate of A-to-G and T-to-C base damage (e.g., due to bisulfite treatment), errors do not appear to be trinucleotide-specific. FIG. 17 thus provides an example of a performance evaluation for feature selection, by assessing the association of specific features (e.g., trinucleotide contexts) with desired outputs (e.g., base labels).

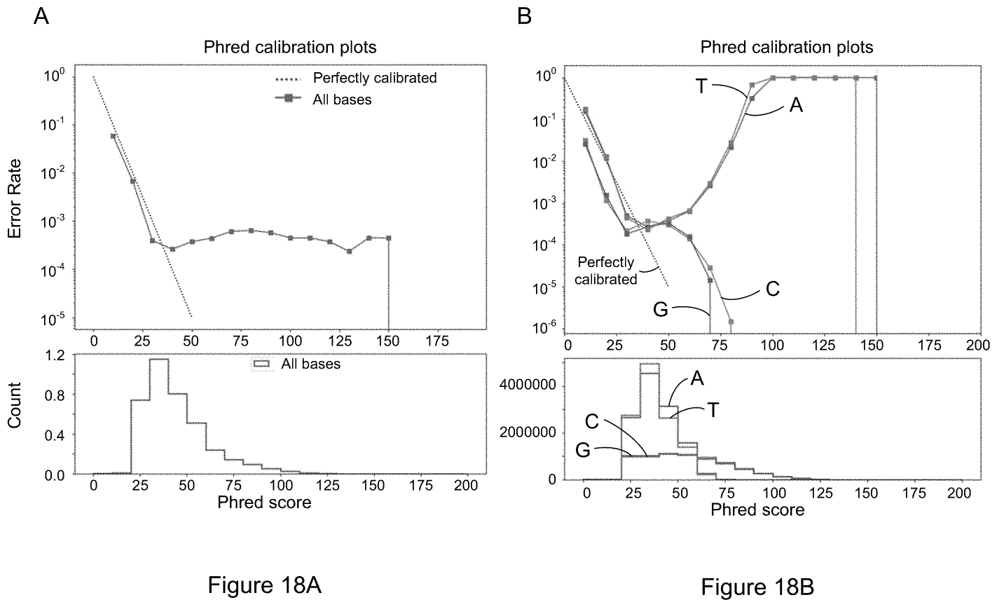

FIG. 18 illustrates the actual error rate for the ML-based collapser compared to an expected, theoretical error rate (e.g., the dashed line). FIG. 18A plots the combined error rate and count of base reads as a function of phred score, for all base classes. Base reads with phred scores of approximately 50 and higher exhibit high actual error rates compared to theoretical error rates, highlighting the need for recalibration of quality scores. FIG. 18B plots the error rate and count of base reads as a function of phred score, for each individual base class. Notably, at phred scores of approximately 50 and higher, A and T bases exhibit extremely high actual error rates compared to theoretical error rates, whereas G and C bases exhibit moderately high actual error rates compared to theoretical error rates. These plots additionally highlight the need for base-specific recalibration of quality scores to account for differences in actual errors rates for individual base classes.

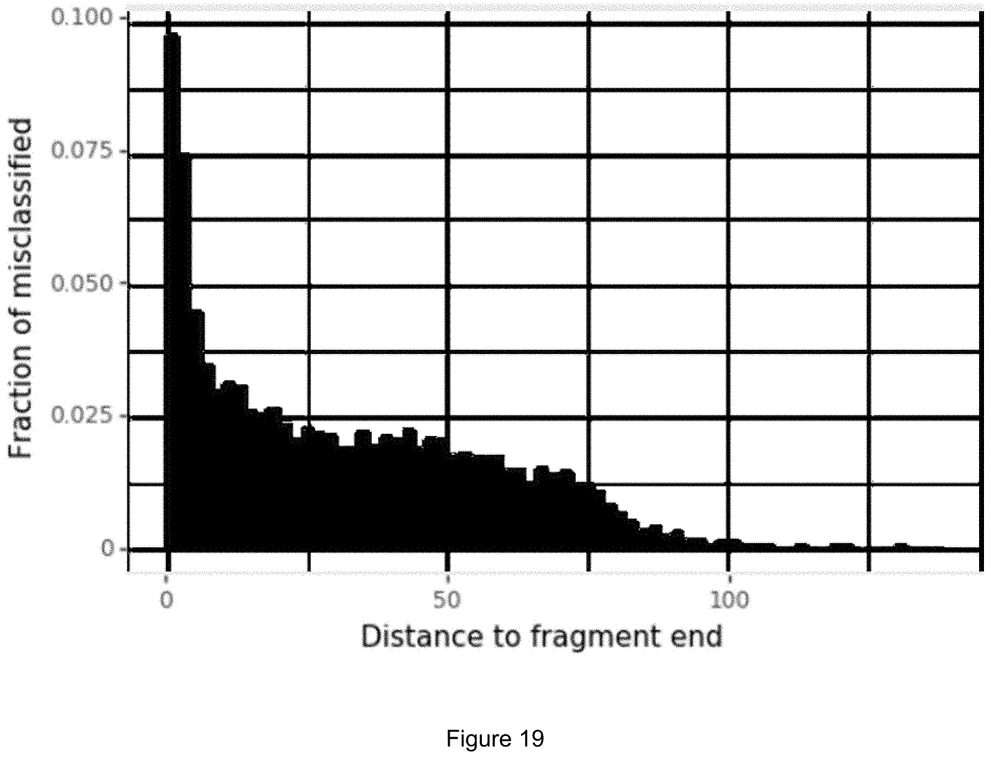

FIG. 19 provides another example of a performance evaluation for feature selection. The figure plots the fraction of misclassified base reads as a function of distance to fragment end, revealing that the distance of a base read to the end of a sequence read is correlated with base call accuracy and is thus an informative feature for model training and classification. The x-axis denotes a symmetric distance to the fragment end (e.g., distance from either end). The four bases closest to the end of a sequence read are shown to have high rates of error, whereas error rates are substantially reduced beyond the first four base positions.

Overall, the performance evaluation of the ML-based collapser on targeted methylation sequencing data revealed that the large majority of bases were easily linearly separable and correctly classified, confirming the robustness of the methods and embodiments disclosed herein. Although the performance of the ML-based collapser was limited at base positions with low bag depths, recalibration of the model improved accuracy by providing realistic quality scores that reflect empirical error rates.

### Example 6

### Performance of ML-Based Collapser Using Whole Genome Bisulfite Sequencing Data

Enzymatically treated whole genome bisulfite sequencing (WGBS) data was collapsed in accordance with some embodiments of the present disclosure. Collapsed samples included two fully methylated datasets and two fully unmethylated datasets, where methylated base positions were assigned a “1” value and unmethylated base positions were assigned a “0” value. Expected results from the ML-based collapser included a correction of collapsed base reads to 0 or 1, particularly for fragments with fractional beta scores (e.g., where a first proportion of the base reads at the respective base position are predicted to be methylated and a second proportion of the base reads at the respective base position are predicted to be unmethylated).

TABLE 4
Fragment Characterization

| | | | | |
|---|---|---|---|---|
| # fragments | β = 1 | 0 < β < 1 | β = 0 | % “correct” |

Uncollapsed

| | | | | | |
|---|---|---|---|---|---|
| Methylated 1 | 3802769 | 2870742 | 2870742 | 1451 | 75.49 |
| Methylated 2 | 3164470 | 2371247 | 792070 | 1153 | 74.93 |
| Unmethylated 1 | 3890939 | 12917 | 1294863 | 2583159 | 66.39 |
| Unmethylated 2 | 3830748 | 13667 | 1284211 | 2532870 | 66.12 |

Collapsed

| | | | | | |
|---|---|---|---|---|---|
| Methylated 1 | 3885978 | 3007995 | 875606 | 2377 | 77.41 |
| Methylated 2 | 3238435 | 2497201 | 739351 | 1883 | 77.11 |
| Unmethylated 1 | 3979604 | 13573 | 1317598 | 2648433 | 66.55 |
| Unmethylated 2 | 3920477 | 14438 | 1309664 | 2596375 | 66.23 |

Table 4 shows a comparison of the fragment characterization for fragments with more than 5 CpG sites after performing a base calling method using uncollapsed base reads and a base calling method using collapsed base reads. For fully methylated datasets, the base calling method using collapsed base reads resulted in a higher percentage of correctly predicted base reads compared to the base calling method using uncollapsed base reads (e.g., 77.41% and 77.11% compared to 75.49% and 74.93%, respectively).

TABLE 5
CpG Characterization

| | | | | |
|---|---|---|---|---|
| # sites | ambiguous | variant | conflict | % error |

Uncollapsed

| | | | | | |
|---|---|---|---|---|---|
| Methylated 1 | 27335932 | 80881 | 258519 | 220830 | 1.75 |
| Methylated 2 | 22829452 | 74433 | 220627 | 195440 | 1.82 |
| Unmethylated 1 | 28039615 | 104361 | 268451 | 174981 | 1.58 |
| Unmethylated 2 | 27534282 | 97131 | 265830 | 193268 | 1.67 |

Collapsed

| | | | | | |
|---|---|---|---|---|---|
| Methylated 1 | 27925604 | 88151 | 319935 | 0 | 1.15 |
| Methylated 2 | 23354112 | 77723 | 272047 | 0 | 1.16 |
| Unmethylated 1 | 28672748 | 105422 | 851542 | 0 | 2.97 |
| Unmethylated 2 | 28171813 | 98360 | 766858 | 0 | 2.72 |

Table 5 shows a comparison of the CpG site characterization for fragments with more than 5 CpG sites after performing a base calling method using uncollapsed base reads and a base calling method using collapsed base reads. For fully methylated datasets, the base calling method using collapsed base reads resulted in a lower percentage of error compared to the base calling method using uncollapsed base reads (e.g., 1.15% and 1.16% compared to 1.75% and 1.82%, respectively).

### Conclusion

Plural instances may be provided for components, operations, or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event (” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

1. A method for determining a consensus base call for a respective base position within a plurality of base positions of a target nucleic acid molecule in a biological sample of a subject, the method comprising:
at a computer system comprising at least one processor and a memory storing at least one program for execution by the at least one processor, the at least one program comprising instructions for:
a) obtaining a respective sequencing dataset corresponding to a plurality of base reads for the respective base position, in electronic form, wherein each base read of the plurality of base reads is captured in a respective sequence read among a plurality of sequence reads of the target nucleic acid molecule, wherein the respective sequencing dataset includes a respective plurality of features for each respective base read of the plurality of base reads for the respective base position, and wherein the respective plurality of features for each respective base read comprises at least two features selected from the group consisting of:
a nucleotide base of the respective base read,
a read quality score associated with the respective base read,
a strand identifier indicating whether the respective sequence read of the respective base read corresponds to a forward or a reverse strand of the target nucleic acid molecule,
a trinucleotide context based on three consecutive nucleotide bases including the respective base read, and
a confidence score associated with the trinucleotide context;
b) transforming the sequencing dataset into a feature tensor that represents a distribution of the plurality of features in the sequencing dataset; and
c) assessing the feature tensor with a classifier to determine the consensus base call for the respective base position, wherein the consensus base call comprises a predicted nucleotide base.

2. The method of claim 1, the method further comprising:
d) repeating the obtaining (a), transforming (b), and assessing (c) for each respective base position within the plurality of base positions of the target nucleic acid molecule, wherein the plurality of base positions of the target nucleic acid molecule is all or a subset of the target nucleic acid molecule.

3. The method of claim 2, wherein the plurality of base positions is at least 10 base positions, at least 100 base positions, at least 1000 base positions, at least 10,000 base positions, at least 100,000 based positions, or at least 1 million base positions.

4. (canceled)

5. The method of claim 3, wherein the method is performed in one hour or less, thirty minutes or less, ten minutes or less, five minutes or less, or 1 minute or less.

6. The method of claim 1, wherein the classifier determines a model quality score for the consensus base call.

7-8. (canceled)

9. The method of claim 6, the method further comprising a recalibrating of the model quality score by a procedure comprising:
identifying a quality bin corresponding to the model quality score, wherein the quality bin is one of a plurality of quality bins defined by model quality scores obtained during training of the classifier with training data; and
interpolating the model quality score based on an actual error rate at the quality bin to generate a recalibrated model quality score, wherein the actual error rate is a misclassification rate of the classifier on the training data.

10. (canceled)

11. The method of claim 1, wherein the feature tensor represents a quantified distribution of the respective plurality of features for each respective base read of the plurality of base reads, wherein the quantified distribution is determined by:
sorting each base read in the plurality of base reads by rank;
generating a value for each base read in accordance with its respective rank; and
representing the value at each base read in the feature tensor.

12. The method of claim 1, wherein the feature tensor represents a quantified distribution of the respective plurality of features for each base read of the plurality of base reads, the respective plurality of features for each base read comprises a respective nucleotide base and a respective read quality score, and the quantified distribution comprises a discounted distribution that is determined by:
ranking the plurality of base reads by order of decreasing respective read quality scores, thereby assigning each base read in the plurality of base reads with a respective rank number “k”;
generating a value for each base read with a discounted value based on its respective rank number “k”; and
representing the discounted value of each base read at an element in the feature tensor that coincides with a respective nucleotide base and a respective read quality score of the base read, wherein:
for each element coinciding with a base read, assigning the element with the discounted value of the base read;
for each element coinciding with a plurality of base reads, assigning the element with a sum of the discounted values of the plurality of base reads; and
for each element coinciding with no base reads, assigning the element with a zero or null value.

13. The method of claim 1, wherein the assessing c) comprises:
computing a plurality of class probabilities based on the feature tensor, wherein the plurality of class probabilities correspond to a set of classes that includes at least four classes selected from the group consisting of adenine, guanine, thymine, cytosine, and uracil; and
determining the consensus base call based on a highest class probability among the class probabilities.

14. The method of claim 1, wherein the classifier comprises a multinomial logistic regression model and a set of model coefficients learned during training of the classifier or wherein the classifier comprises a multivariate logistic regression model, a neural network, a convolutional neural network, a support vector machine, a Naïve Bayes algorithm, a nearest neighbor algorithm, a random forest algorithm, a decision tree algorithm, a boosted tree algorithm, a regression algorithm, a logistic regression algorithm, a multi-category logistic regression algorithm, a linear discriminant analysis algorithm, or a supervised clustering model.

15. The method of claim 1, the method further comprising using a penalized logistic regression model to derive a plurality of model coefficients for training the classifier using the selected at least two features, wherein the penalized logistic regression model is selected at least in part based on principal component analysis of training data, and wherein somatic and germline mutations are removed from the training data.

16-17. (canceled)

18. The method of claim 1, wherein the sequencing dataset further comprises one or more additional features selected from the group consisting of:
a bag depth count of a total number of base reads at the respective base position,
a first bag depth indicating when the total number of base reads is below a minimum threshold,
a distance of a base read relative to an end of a respective sequence read in the plurality of sequence reads,
a terminal base indicating whether the base read is an edge or a non-edge base,
a duplex status indicating whether the base read is associated with a duplex or non-duplex read of the respective base position,
a certain trinucleotide context indicating when the base read is one of three predetermined adjacent nucleotide bases,
an overlap status indicating whether the base read is a stitched or non-stitched read,
an ambiguous strand base count at the base read,
a mapping quality of the base read,
a distance of the base read from a homopolymer, insertion, or deletion,
one or more interaction features between or within duplex strands, and
a read direction indicating whether the respective sequence read of the base read is a forward or reverse read of the target nucleic acid molecule,
and wherein the transforming b) comprises representing values associated with the additional features at one or more elements appended to the feature tensor.

19-21. (canceled)

22. The method of claim 1, wherein the target nucleic acid molecule is a cell-free nucleic acid molecule.

23. The method of claim 1, wherein the sequencing dataset is obtained from a methylation sequencing or a targeted sequencing.

24. (canceled)

25. The method of claim 1, further comprising, prior to obtaining (a) the sequencing dataset, obtaining a mapping string for each sequence read in a plurality of sequence reads of the target nucleic acid molecule, thereby obtaining a plurality of mapping strings, wherein each mapping string in the plurality of mapping strings:
(i) is determined by an alignment of the respective sequence read to a reference genome; and
(ii) comprises a plurality of encodings, wherein each encoding in the plurality of encodings represents a coordinate match status of one or more base positions in the respective sequence read compared with the reference genome,
wherein each encoding in the plurality of encodings is selected from the group consisting of match, insertion, deletion, skipped, soft-clipping, and hard-clipping, or
wherein each encoding in the plurality of encodings further comprises an indication of a number of base positions in the one or more base positions that have the respective coordinate match status.

26-27. (canceled)

28. The method of claim 1, wherein:
each base position in the plurality of base positions of the target nucleic acid molecule that fails to satisfy a selection criterion is removed from one or more sequence reads in the plurality of sequence reads, and
the selection criterion is a measure of central tendency of the number of observations of one or more encodings for the respective base position, across each sequence read in the plurality of sequence reads that align to a region of the reference genome spanning at least the respective base position.

29. (canceled)

30. The method of claim 1, further comprising removing from the plurality of sequence reads of the target nucleic acid molecule each base position in the plurality of base positions that satisfies a filtering criterion,
wherein the filtering criterion is satisfied when the plurality of base reads comprises a threshold number of alternative nucleotide base identities at the respective base position, or
wherein the filtering criterion is satisfied when, for each respective sequence read in the plurality of sequence reads, the respective base position comprises a positive strand nucleotide base identity of cytosine and a negative strand nucleotide base identity of guanine.

31-32. (canceled)

33. The method of claim 1, wherein the plurality of sequence reads provides an average coverage of between 20× and 70,000× at the base position.

34. (canceled)

35. A non-transitory computer readable storage medium storing one or more programs for consensus base calling for a respective base position within a plurality of base positions of a target nucleic acid molecule in a biological sample of a subject, the one or more programs configured for execution by a computer, the one or more programs comprising instructions for:
a) obtaining a respective sequencing dataset corresponding to a plurality of base reads for the respective base position, in electronic form, wherein each base read of the plurality of base reads is captured in a respective sequence read among a plurality of sequence reads of the target nucleic acid molecule, wherein the respective sequencing dataset includes a respective plurality of features for each respective base read of the plurality of base reads for the respective base position, and wherein the respective plurality of features for each respective base read comprises at least two features selected from the group consisting of:
a nucleotide base of the respective base read,
a read quality score associated with the respective base read,
a strand identifier indicating whether the respective sequence read of the respective base read corresponds to a forward or a reverse strand of the target nucleic acid molecule,
a trinucleotide context based on three consecutive nucleotide bases including the respective base read, and
a confidence score associated with the trinucleotide context;
b) transforming the sequencing dataset into a feature tensor that represents a distribution of the plurality of features in the sequencing dataset; and
c) assessing the feature tensor with a classifier to determine the consensus base call for the respective base position, wherein the consensus base call comprises a predicted nucleotide base.

36. (canceled)

37. A computer system, comprising:
one or more processors;
memory storing one or more programs to be executed by the one or more processors;
the one or more programs comprising instructions for consensus base calling for a respective base position within a plurality of base positions of a target nucleic acid molecule in a biological sample of a subject by a method comprising:
a) obtaining a respective sequencing dataset corresponding to a plurality of base reads for the respective base position, in electronic form, wherein each base read of the plurality of base reads is captured in a respective sequence read among a plurality of sequence reads of the target nucleic acid molecule, wherein the respective sequencing dataset includes a respective plurality of features for each respective base read of the plurality of base reads for the respective base position, and wherein the respective plurality of features for each respective base read comprises at least two features selected from the group consisting of:
a nucleotide base of the respective base read,
a read quality score associated with the respective base read,
a strand identifier indicating whether the respective sequence read of the respective base read corresponds to a forward or a reverse strand of the target nucleic acid molecule,
a trinucleotide context based on three consecutive nucleotide bases including the respective base read, and
a confidence score associated with the trinucleotide context;
b) transforming the sequencing dataset into a feature tensor that represents a distribution of the plurality of features in the sequencing dataset; and
c) assessing the feature tensor with a classifier to determine the consensus base call for the respective base position, wherein the consensus base call comprises a predicted nucleotide base.

38. (canceled)
